(19) United States
(12) Patent Application Publication
(10) Pub. No.: US 2026/0306385 A1
(43) Pub. Date: Oct. 8, 2026

(54) COSMETIC COMPOSITION COMPRISING AN ENDOLYSIN AND AN ORGANIC FILLER

(71) Applicant: L'OREAL, Paris (FR)

(72) Inventors: Eric DUPONCHEL, Chevilly Larue (FR); Toni MOINE, Chevilly Larue (FR)

(73) Assignee: L'OREAL, Paris (FR)

(21) Appl. No.: 19/484,670

(22) PCT Filed: May 31, 2024
(86) PCT No.: PCT/EP2024/065124

§ 371 Date: Nov. 14, 2025

(30) Foreign Application Priority Data
Jun. 2, 2023 (FR) FR2305556

(51) Int. Cl. A61K 8/66; A61K 8/44; A61K 8/73; A61K 38/47; A61K 47/18; A61K 47/36; A61P 31/04; A61Q 17/00

(52) U.S. Cl. CPC A61K 8/66; A61K 8/44; A61K 8/732; A61K 38/47; A61K 47/183; A61K 47/36; A61P 31/04; A61Q 17/005

(57) ABSTRACT

The present invention relates to a composition, notably a cosmetic composition, comprising, in a physiologically acceptable medium, at least one endolysin, in particular an endolysin derived from a Staphylococcus aureus phage, and at least one organic filler chosen from an unmodified starch, an N-acylamino acid, salts thereof and mixtures thereof.

The invention also relates to the use of such a composition for preventing and/or treating a skin disorder linked to colonization by Staphylococcus aureus in an individual in need thereof, and in particular for preventing and/or treating acne and/or eczema in an individual in need thereof, and to a non-therapeutic cosmetic process for caring for keratin materials, in particular the skin, comprising at least one step of topical application to said keratin materials of such a composition.

### TECHNICAL FIELD

The present invention relates to a composition, notably a cosmetic composition, comprising, in a physiologically acceptable medium, at least one endolysin, in particular an endolysin derived from a Staphylococcus aureus phage, and at least one organic filler chosen from an unmodified starch, an N-acylamino acid, salts thereof and mixtures thereof.

The invention also relates in particular to the use of a composition of the invention for preventing and/or treating a skin disorder linked to colonization by a bacterium of the Staphylococcus aureus species, and in particular for preventing and/or treating acne and/or eczema in an individual in need thereof.

Finally, the invention relates to a non-therapeutic cosmetic process for caring for keratin materials, in particular the skin, comprising the topical application to these keratin materials of a composition according to the invention.

### PRIOR ART

Human skin is permanently populated by a multitude of different microorganisms (bacteria, yeasts and fungi). The resident microbial flora, which is essential to the good health of the skin, consists mainly of propionibacteria (Cutibacterium acnes), staphylococci (Staphylococcus epidermidis and Staphylococcus hominis), corynebacteria and streptococci, and also of a fungal flora composed mainly of Malassezia.

Certain dermatological disorders are usually due to the disruption of the ecological balance of the resident flora following a preponderant colonization of opportunistic microorganisms that are not beneficial to the skin, such as Staphylococcus aureus, which is known to be associated with atopic dermatitis (eczema), greasy or hyperseborrhoeic skin, and acne. To counteract this excess colonization by these opportunistic microorganisms that are not beneficial to the skin, it is common practice to use broad-spectrum antimicrobials or bacteriostats. However, the use of these compounds poses the problem of non-specificity of action targeting both the undesirable opportunistic flora and the resident beneficial flora, and the problem of the risk of the appearance of bacterial resistance or imbalances by selecting resistant bacteria, and also of problems of skin tolerance (irritation, allergies, etc.).

There is thus a need to find novel compounds with good antimicrobial efficacy which do not have the abovementioned drawbacks, and which have good skin tolerance.

It has thus been demonstrated that endolysins from Staphylococcus aureus phages (i.e. phages infecting Staphylococcus aureus) can specifically target S. aureus and lyse, and thus specifically destroy, this bacterium while preserving the resident skin flora (WO 2012/150858 A1). However, it is well known that one of the main obstacles to the application of endolysins targeting Staphylococcus species is a problem of the stability of these proteins and/or of their enzymatic activity, notably the maintenance of this activity over time.

Indeed, it has notably been observed that the lytic action of these enzymes is significantly impaired by interactions with raw materials, in particular when the endolysin is introduced into formulations, notably cosmetic formulations.

There is thus a need for an organic filler that allows the antimicrobial activity of endolysins used in formulations, notably cosmetic formulations, to be preserved over time.

There is moreover a need for an organic filler designed (i) to help maintain over time the antimicrobial activity of endolysins used in compositions, notably cosmetic compositions, (ii) while at the same time preserving the specificity of these endolysins for the target bacteria, in particular Staphylococcus aureus in the present invention.

There is also a need for an organic filler designed to:
help maintain over time the antimicrobial activity of endolysins used in compositions, notably cosmetic compositions;
while at the same time preserving the specificity of these endolysins for the target bacteria, in particular Staphylococcus aureus in the present invention; and
while at the same time affording the composition good sensory properties.

### DISCLOSURE OF THE INVENTION

The aim of the present invention is to solve at least one abovementioned technical problem. Specifically, the inventors have now discovered that native corn starch and lauroyl lysine, compared with other mineral or organic fillers known in the formulation field, in particular cosmetic formulation, prove to be capable of maintaining good Staphylococcus aureus killing performance by the associated endolysin one week after the preparation of a composition comprising same.

### SUMMARY OF THE INVENTION

As mentioned above, the present invention thus relates to a composition, notably a cosmetic composition, comprising, in a physiologically acceptable medium:
(i) at least one endolysin, in particular an endolysin derived from a Staphylococcus aureus phage; and
(ii) at least one organic filler chosen from an unmodified starch, an N-acylamino acid, salts thereof and mixtures thereof.

As illustrated in the examples below, the Applicant has discovered, surprisingly, that a composition according to the invention, comprising an endolysin, in particular an endolysin derived from a Staphylococcus aureus phage, and a native corn starch or lauroyl lysine, advantageously enables the S. aureus killing performance of the endolysin to be maintained. Thus, the present invention also relates to the use of a composition of the invention for preventing and/or treating a skin disorder linked to colonization by a bacterium of the Staphylococcus aureus species, and in particular for preventing and/or treating acne and/or eczema in an individual in need thereof.

The invention moreover relates to a non-therapeutic cosmetic process for caring for keratin materials, in particular the skin, comprising the topical application to these keratin materials of a composition according to the invention.

### DETAILED DESCRIPTION

For the purposes of the present invention, the term “organic filler” is intended to denote colourless or white organic, natural or synthetic solid particles of any form, which are in a form that is insoluble and dispersed in the medium of the composition.

The term “cosmetic” means a composition that is compatible with keratin materials, in particular the skin, mucous membranes and the integuments, more particularly the skin. The composition according to the invention is non-therapeutic.

The term “keratin materials” is intended to denote in particular the skin, mucous membranes, fibres, eyelashes and the integuments.

The term “the skin” means all the skin of the body, and preferably the skin of the face, the scalp, the neckline, the neck, the arms and forearms, the eyelids, around the mouth or behind the ears, the hollow of the elbow, the back of the knees, the hands, the wrists and the ankles, or even more preferably the skin of the face (in particular the forehead, the nose, the cheeks and the chin), the neckline and the neck.

A composition according to the invention comprises a physiologically acceptable medium, i.e. one which has a pleasant colour, odour and feel and which does not give rise to any unacceptable discomfort, i.e. tingling, tautness or redness, that is liable to discourage the user from applying this composition. Needless to say, a person skilled in the art will take care to choose a physiologically acceptable medium such that the advantageous properties of the endolysin(s) of the invention are not, or are not substantially, adversely affected. Thus, by way of illustration, a physiologically acceptable medium may consist mainly of water and/or of one or more water-miscible organic solvent(s).

A physiologically acceptable medium according to the invention preferentially has a pH between 4 and 8, more particularly between 4.5 and 7.5. Thus, a composition according to the invention may comprise one or more pH adjusters. According to a preferred embodiment, a composition according to the invention comprises at least one pH adjuster.

As used herein, the terms “treat” and “treatment” are intended to denote the alleviation of the symptoms associated with a specific disorder or condition and/or the elimination of said symptoms and also the complete disappearance of the disorder or condition in question.

In the context of the present invention, the terms “prevent” and “prevention” denote the reduction, to a lesser degree, of the risk or probability of occurrence of a given phenomenon.

### Endolysin

A composition, notably a cosmetic composition, according to the invention is first characterized in that it comprises at least one endolysin.

In the embodiments described herein, the endolysin may be a native bacteriophage endolysin or a recombinant endolysin and may be any endolysin known to those skilled in the art. As used herein, the terms bacteriophage lysin, bacteriophage endolysin and endolysin are used interchangeably. An endolysin may be chosen from the group of endolysins defined in documents WO 2011/023702, WO 2012/146738, WO 2003/082184, WO 2010/011960, WO 2010/149795, WO 2010/149792, WO 2012/094004, WO 2011/023702, WO 2011/065854, WO 2011/076432, WO 2011/134998, WO 2012/059545, WO 2012/085259, WO 2012/146738, WO 2018/091707, Exebacase™ (Lysin CF-301); SAL200™ or Tonabacase; Auresine™ (Sigma-Aldrich SAE0083), and Ectolysin™ P128.

In the embodiments presented herein, the endolysin is a Staphylococcus aureus-specific endolysin, i.e. it will effectively lyse Staphylococcus aureus but will not substantially lyse bacteria other than Staphylococcus aureus. In particular, an endolysin used according to the invention will lyse Staphylococcus aureus, but not Staphylococcus epidermidis.

Most native Staphylococcus bacteriophage endolysins with peptidoglycan hydrolase activity, such as endolysin Ply2638, are composed of a C-terminal cell wall binding domain (CBD), a central N-acetylmuramoyl-L-alanine amidase domain and an N-terminal alanyl-glycyl endopeptidase domain with cysteine, in the case of Ply2638, an N-terminal alanyl-glycine endopeptidase domain with peptidase_M23 homology, the latter three domains each exhibiting peptidoglycan hydrolase activity with a different target-binding specificity and in general referred to as enzymatically active domains.

The endolysin may be a recombinant endolysin, such as a recombinant Staphylococcus aureus-specific endolysin, in particular a recombinant chimeric Staphylococcus aureus-specific endolysin comprising one or more heterologous domains.

In general, endolysins consist of different subunits (domains), for example a cell wall binding domain (CBD) and one or more enzymatic domains with peptidoglycan activity, such as an amidase domain, an M23 domain and a CHAP domain (cysteine, histidine-dependent amidohydrolases/peptidases). An example of a chimeric endolysin specific for Staphylococcus aureus comprising one or more heterologous domains is an endolysin comprising an amidase domain of bacteriophage Ply2638, an M23 domain of lysostaphin (S. simulans) and a cell wall-binding domain of bacteriophage Ply2638.

This Staphylococcus aureus-specific chimeric endolysin is a preferred endolysin and is described in detail in WO 2012/150858. Other preferred endolysins are described in detail in WO 2013/169104. Other preferred endolysins are described in detail in WO 2016/142445. Other preferred endolysins according to the invention are widely described in WO 2017/046021. Other preferred endolysins according to the invention are widely described in WO 2012/146738, WO 2003/082184, WO 2010/011960, WO 2010/149795, WO 2011/076432, WO 2011/134998, WO 2012/085259, WO 2012/146738 and WO 2018/091707.

An endolysin used according to the invention may comprise a domain having at least 80% sequence identity with a domain described in WO 2012/150858, WO 2013/169104, WO 2016/142445, WO 2017/046021, WO 2012/146738, WO 2003/082184, WO 2010/011960, WO 2010/149795, WO 2011/076432, WO 2011/134998, WO 2012/085259, WO 2012146738 or WO 2018/091707.

An endolysin used according to the invention may have at least 80% sequence identity with an endolysin described in WO 2012/150858, WO 2013/169104, WO 2016/142445, WO 2017/046021, WO 2012/146738, WO 2003/082184, WO 2010/011960, WO 2010/149795, WO 2011/076432, WO 2011/134998, WO 2012/085259, WO 2012/146738, WO 2018/091707, such as endolysin with the amino acid sequence of reference SEQ ID NO: 29 in WO 2012/150858.

In one particular embodiment, the endolysin is an endolysin derived from a Staphylococcus aureus phage.

For the purposes of the present invention, the term “endolysin derived from a Staphylococcus aureus phage” means a native or recombinant protein such as an enzyme or nucleic acid molecule encoding for same derived from one or more bacteriophage(s) that are capable of lysing the wall of bacteria of the species Staphylococcus aureus.

The endolysin notably comprises one or more domains for binding to the bacterial wall of Staphylococcus aureus and/or one or more domains for lysing the bacterial wall of Staphylococcus aureus, said binding domain(s) and lysis domain(s) for the Staphylococcus aureus bacterial wall being derived from one or more identical or different bacteriophage(s) that are capable of lysing the wall of bacteria of the species Staphylococcus aureus.

The endolysin used in the context of the present invention may be in native or recombinant form, in particular in recombinant form.

According to a particular embodiment, the endolysin comprises a first protein sequence comprising a domain for binding to the cell wall of species of the genus Staphylococcus.

In particular, the first protein sequence is derived from the endolysin of the Φ2638a bacteriophage of S. aureus.

For each of the amino acid or nucleic acid sequences of interest, reference sequences are described herein. The present description also encompasses amino acid or nucleic acid sequences (for example enzyme amino acid sequences), having specific percentages of amino acid or nucleotide identity with a reference sequence.

For obvious reasons, throughout the present description, a specific nucleic acid sequence or a specific amino acid sequence which respects, respectively, the nucleotide or amino acid identity under consideration, must also lead to the production of a protein (or enzyme) which displays the desired biological activity. As used herein, the “percentage identity” between two nucleic acid sequences or between two amino acid sequences is determined by comparing the two optimally aligned sequences through a comparison window.

The portion of the nucleotide or amino acid sequence in the comparison window may thus comprise additions or deletions (e.g. “gaps”) relative to the reference sequence (which does not comprise these additions or deletions) so as to achieve an optimal alignment between the two sequences.

The terms “sequence homology” or “sequence identity” or “homology” or “identity” are used interchangeably herein. For the purposes of the invention, this means that in order to determine the percentage of sequence homology or sequence identity of two amino acid sequences or two nucleic acid sequences, the sequences are aligned for optimal comparison. In order to optimize the alignment between the two sequences, gaps may be introduced in either of the two sequences being compared. This alignment may be performed over the entire length of the sequences being compared. The alignment may also be performed over a shorter length, for example over about twenty, fifty, one hundred or more nucleic acids/bases or amino acids. Sequence identity is the percentage of identical matches between the two sequences over the reported aligned region.

The comparison of sequences and the determination of the percentage of sequence identity between two sequences may be performed using a mathematical algorithm. A person skilled in the art knows that several different computer programs are available for aligning two sequences and determining the identity between two sequences (Kruskal, J. B. (1983) An overview of sequence comparison In D. Sankoff and J. B. Kruskal, (ed.), Time warps, string edits and macromolecules: the theory and practice of sequence comparison, pages 1-44 Addison Wesley).

The percentage of sequence identity between two amino acid sequences or between two nucleotide sequences may be determined using the Needleman-Wunsch algorithm for the alignment of two sequences. (Needleman, S. B. and Wunsch, C. D. (1970) J. Mol. Biol., 48, 443-453). The algorithm allows the alignment of both amino acid sequences and nucleotide sequences. The Needleman-Wunsch algorithm was implemented in the NEEDLE computer program.

For the purposes of the invention, the NEEDLE program of the EMBOSS software package was used (version 2.8.0 or higher, EMBOSS: The European Molecular Biology Open Software Suite (2000) Rice, P. Longden, J. and Bleasby, A. Trends in Genetics 16, (6) pages 276-277, http://emboss.bioinformatics.nl/). For protein sequences, EBLOSUM62 is used for the substitution matrix. For nucleotide sequences, EDNAFULL is used. The optional parameters used are a space opening penalty of 10 and a space extension penalty of 0.5. No end gap penalty is added. In the Output section, Yes has been indicated in response to the question “Brief identity and similarity” and “SRS pairwise” has been indicated as the output alignment format.

After alignment by the NEEDLE program described above, the percentage of sequence identity between a query sequence and a sequence of the invention is calculated as follows: Number of matching positions in the alignment showing an identical amino acid or nucleotide in the two sequences divided by the total length of the alignment after subtracting the total number of gaps in the alignment. The identity defined here may be obtained from NEEDLE using the NOBRIEF option and is labelled in the output of the program as “longest identity”.

The similarity of nucleotide and amino acid sequences, i.e. the percentage identity of the sequences, may be determined by sequence alignments using several other known algorithms, preferably the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available, for example, at https://www.ebi.ac.uk/Tools/msa/clustalo/or the GAP program (mathematical algorithm of the University of Iowa) or the mathematical algorithm of Myers and Miller (1989—Cabios 4:11-17) or Clone Manager 9. The preferred parameters used are the default parameters as defined at https://www.ebi.ac.uk/Tools/msa/clustalo/.

The degree of sequence identity (sequence matching) may be calculated using, for example, BLAST, BLAT or BlastZ (or BlastX). A similar algorithm is incorporated in the BLASTN and BLASTP programs of Altschul et al. (1990) J. Mol. Biol., 215, 403-410. BLAST polynucleotide searches are performed with the BLASTN program, score=100, word length=12, so as to obtain polynucleotide sequences homologous to the nucleic acids which encode the protein of interest.

BLAST protein searches are performed with the BLASTP program, score=50, word length=3, to obtain amino acid sequences homologous to the SHC polypeptide. To obtain gapped alignments for comparison, Gapped BLAST is used as described in Altschul et al. (1997) Nucleic Acids Res. 25, 3389-3402. When using the BLAST and Gapped BLAST programs, the default settings of the respective programs are used. Sequence matching analysis may be complemented by established homology mapping techniques such as Shuffle-LAGAN (Brudno M., Bioinformatics 2003b, 19 Suppl. 1:154-162) or Markov random fields. Where reference is made to percentages of sequence identity in the present patent application, these percentages are calculated relative to the total length of the longest sequence, unless otherwise indicated.

In particular embodiments, the percentage identity between two sequences is determined using CLUSTAL O (version 1.2.4).

Thus, according to a particular embodiment, the first protein sequence comprises a protein sequence comprising at least 80%, in particular at least 90%, more particularly at least 95% sequence identity with the amino acid sequence of reference SEQ ID NO: 1.

The term “at least 80% sequence identity between two sequences” means that the first sequence may comprise 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the second sequence, whether they are amino acid sequences or nucleic acid sequences.

In particular, the first protein sequence consists of an amino acid sequence of reference SEQ ID NO: 1.

The protein sequences described herein may be encoded by one or more allelic variants.

An allelic variant denotes any one of two or more alternative forms of a gene occupying the same chromosomal locus. A preferred nucleic acid variant is a nucleotide sequence that contains one or more silent mutations. Alternatively or in combination, a nucleic acid variant may also be obtained by introducing nucleotide substitutions, which do not result in another amino acid sequence of the polypeptide encoded by the nucleotide sequence, but which correspond to the use of codons of the host organism intended for the production of the polypeptide of the invention. According to a preferred embodiment, a nucleic acid variant encodes a polypeptide still having its biological function. More preferably, a nucleotide sequence variant encodes a polypeptide displaying binding to the cell wall of species of the genus Staphylococcus and/or lytic activity. Even more preferably, a nucleic acid variant encodes a polypeptide displaying increased binding to the cell wall of species of the genus Staphylococcus and/or increased lytic activity, as defined hereinbelow. Nucleic acids encoding a polypeptide displaying binding to the cell wall of species of the genus Staphylococcus and/or lytic activity may be isolated from any microorganism.

All these variants may be obtained using techniques known to those skilled in the art, such as library screening by hybridization (Southern blot procedures) under low to medium to high hybridization conditions. Low to medium to high stringency conditions means pre-hybridization and hybridization at 42° C. in 5×SSPE, 0.3% SDS, 200 μg/ml sheared and denatured salmon sperm DNA, and either 25%, 35% or 50% formamide for low to medium to high stringencies, respectively. Next, the hybridization reaction is washed three times for 30 minutes using for each wash 2×SSC, 0.2% SDS and at 55° C., 65° C. or 75° C. for low to medium to high stringencies.

According to a particular embodiment, the first protein sequence is encoded by a nucleic acid sequence comprising at least 80%, in particular at least 90%, more particularly at least 95% sequence identity with the nucleic acid sequence of reference SEQ ID NO: 2.

In particular, the first protein sequence is encoded by a nucleic acid sequence consisting of a nucleic acid sequence of reference SEQ ID NO: 2.

The binding of a domain to the peptidoglycan cell wall of Staphylococcus genera may be evaluated using assays that are well known to those skilled in the art. In a preferred embodiment, an immunohistochemical technique and/or a gene fusion technique resulting in labelled constructs of either domain are used to evaluate the specific binding of peptides, polypeptides or proteins to the peptidoglycan cell wall of Staphylococcus genera. The signal quantification processes used in the abovementioned immunohistochemical or fusion techniques are well known in the art.

In one embodiment, the binding to the peptidoglycan cell wall of Staphylococcus may be quantified using a fluorescent fusion construct comprising a polypeptide comprising a domain included in a first protein sequence as described previously. Such a cell wall binding assay is described in detail by Loessner et al. (Molecular Microbiology 2002, 44 (2): 335-349). In said assay, a solution comprising said fluorescent fusion construct or a negative control, preferably green fluorescent protein (GFP), is subjected to Staphylococcus cells, preferably S. aureus cells, more preferably S. aureus BB255, for a specified period of time, after which the cells are sedimented by centrifugation together with the bound fluorescent fusion constructs. The fluorescent signal of Staphylococcus cells exposed to a fluorescent fusion construct, subtracted from the fluorescent signal of Staphylococcus cells exposed to a negative control, preferably GPF, is a measure of cell binding for the purposes of the present invention.

Examples of evaluation of the binding of an endolysin to the cell wall of species of the genus Staphylococcus are notably illustrated in WO 2012/150858 A1.

Preferably, in the context of the present text, a protein sequence will be said to comprise a domain for binding to the peptidoglycan cell wall of Staphylococcus genera when, using this assay, an increase in the fluorescent signal of the sedimented cells is detected. The binding is preferably said to be specific. Preferably, an endolysin is described comprising a domain which displays a binding capacity, as defined herein, of at least 50, 60, 70, 80, 90 or 100, 150 or 200% of the peptidoglycan cell wall binding of the Φ2638a bacteriophage endolysin of S. aureus (Ply2638) encoded by the nucleic acid sequence of reference SEQ ID NO: 5.

According to a particular embodiment, the binding activity to the cell wall of species of the genus Staphylococcus is measured by an immunohistochemical technique and/or a gene fusion technique, in particular a fluorescent fusion technique, more particularly fusion with a green fluorescent protein.

According to a particular embodiment, the endolysin comprises a protein sequence comprising at least 80%, in particular at least 90%, more particularly at least 95% sequence identity with an amino acid sequence chosen from the group consisting of the amino acid sequences of references SEQ ID NO: 3 and SEQ ID NO: 4.

According to a particular embodiment, the endolysin comprises a protein sequence encoded by a nucleic acid sequence comprising at least 80%, in particular at least 90%, more particularly at least 95% sequence identity with the nucleic acid sequence of reference SEQ ID NO: 5.

In particular, the protein sequence may be encoded by a nucleic acid sequence consisting of a nucleic acid sequence of reference SEQ ID NO: 5.

According to a particular embodiment, the endolysin may also comprise a heterologous protein sequence.

The term “heterologous protein sequence” is intended to denote a protein sequence, i.e. an amino acid sequence or a nucleic acid sequence encoding the protein sequence, which is not naturally functionally linked as a neighbouring sequence to said first protein sequence. As used herein, the term “heterologous” may mean “recombinant”. The term “recombinant” refers to a genetic entity different from that generally found in nature. When applied to a nucleotide sequence or nucleic acid molecule, this means that said nucleotide sequence or nucleic acid molecule is the product of various combinations of cloning, restriction and/or ligation steps, and other procedures that result in the production of a construct that is different from a sequence or molecule found in nature.

Such protein or nucleic acid recombination methods are well known to those skilled in the art.

According to a particular embodiment, the endolysin comprises a heterologous protein sequence comprising a lytic domain. Preferably, said lytic domain shows peptidoglycan hydrolase activity.

“Peptidoglycan hydrolase activity”, also defined herein as “lytic activity”, may be evaluated via processes that are well known to those skilled in the art. In one embodiment, the lytic activity may be evaluated spectrophotometrically by measuring the decrease in turbidity of substrate cell suspensions. Preferably, the lytic activity may be evaluated spectrophotometrically by measuring the decrease in turbidity of a suspension of S. aureus, the turbidity being quantified by measuring the OD₅₉₅ spectrophotometrically (Libra S22, Biochrom). More preferably, 200 nM of a polypeptide encoded by a nucleic acid molecule as identified herein are incubated with a suspension of S. aureus having an initial OD₆₀₀ of 1±0.05, as evaluated spectrophotometrically (Libra S22, Biochrom), in PBS buffer pH 7.4, 120 mM sodium chloride for 30 min at 37° C. The decrease in turbidity is calculated by subtracting the OD₅₉₅ after 30 min of incubation from the OD₅₉₅ before 30 min of incubation. In the context of the present text, a protein sequence will be said to comprise a lytic domain when, using this assay, a decrease in turbidity of at least 10, 20, 30, 40, 50 or 60% is detected. Preferably, a decrease of at least 70% is detected. Preferably, an endolysin is described comprising a domain which shows lytic activity of at least 50, 60, 70, 80, 90, 100, 150 or 200% or more of a lytic activity of the Φ2638a bacteriophage endolysin of S. aureus (Ply2638) encoded by the nucleic acid sequence of reference SEQ ID NO: 5.

According to a particular embodiment, the lytic activity of the endolysin is measured spectrophotometrically by measuring the decrease in turbidity of a suspension of S. aureus.

In one embodiment, the endolysin is possibly not encoded by an amino acid sequence comprising or consisting of an amino acid sequence chosen from the group consisting of the amino acid sequences of references SEQ ID NO: 3 and SEQ ID NO: 4.

In particular, the endolysin is possibly not encoded by a nucleic acid sequence comprising or consisting of the nucleic acid sequence of reference SEQ ID NO: 5, encoding the Φ2638 bacteriophage endolysin of S. aureus.

According to a particular embodiment, the heterologous protein sequence comprises a lytic domain, said lytic domain comprising a second and a third protein sequence, said second protein sequence comprising an endopeptidase M23 domain and said third protein sequence comprising an amidase domain.

An endopeptidase domain as used herein preferably cleaves pentaglycine cross-bridges (Trayer, H. R. and Buckley, C. E. (1970) Molecular properties of lysostaphin, a specific bacteriolytic agent for Staphylococcus aureus. J. Biol. Chem. 245, 4842-4846) which are found in the cell wall of Staphylococcus genera, preferably in the cell wall of S. aureus, S. simulans and S. carnosus.

An amidase domain as used herein preferably hydrolyses substrates containing gamma-glutamyl.

The functionality and activity of such domains in a polypeptide may be confirmed by characterizing the cleavage products upon incubation of said polypeptides containing any of these domains with a purified peptidoglycan.

According to a particular embodiment, the endopeptidase and/or amidase activity of the endolysin is measured by characterizing the cleavage products.

According to a particular embodiment, the endopeptidase and/or amidase activity of the endolysin may be measured by measuring the optical density of bacteria in the presence of the endolysin. Such methods are notably described in Park et al. (Characterization of an endolysin, LysBPS13, from a Bacillus cereus bacteriophage, FEMS Microbiol. Lett. 2012 July; 332 (1): 76-83) and in Grishin et al. (A Simple Protocol for the Determination of Lysostaphin Enzymatic Activity, Antibiotics (Basle). 2020 Dec. 17; 9 (12): 917).

Preferably, each of the protein sequences and nucleotide sequences encoding the second or third domain is of bacterial or bacteriophage origin.

According to a particular embodiment, said second and third protein sequences are derived, independently of each other, from an enzyme chosen from the group consisting of the endolysin from Φ2638a bacteriophage from S. aureus and the lysostaphin from S. simulans. In particular, one of the second and third protein sequences is derived from the endolysin of Φ2638a bacteriophage of S. aureus and the other sequence of the second and third protein sequences is derived from the lysostaphin of S. simulans.

According to a particular embodiment, said second protein sequence comprises at least 80%, in particular at least 90%, more particularly at least 95% sequence identity with the amino acid sequence of reference SEQ ID NO: 6 and said third protein sequence comprises at least 80%, in particular 90%, more particularly 95% sequence identity with the amino acid sequence of reference SEQ ID NO: 8.

According to a particular embodiment, said second protein sequence is encoded by a nucleic acid sequence comprising at least 80%, in particular at least 90%, more particularly at least 95% sequence identity with the nucleic acid sequence of reference SEQ ID NO: 7 and said third protein sequence is encoded by a nucleic acid sequence comprising at least 80%, in particular at least 90%, more particularly at least 95% sequence identity with the nucleic acid sequence of reference SEQ ID NO: 9.

According to a particular embodiment, said second protein sequence is encoded by a nucleic acid sequence consisting of the nucleic acid sequence of reference SEQ ID NO: 7 and said third protein sequence is encoded by a nucleic acid sequence consisting of the nucleic acid sequence of reference SEQ ID NO: 9.

According to a particular embodiment, the endolysin comprises a protein sequence comprising at least 80%, in particular at least 90%, more particularly at least 95% sequence identity with an amino acid sequence chosen from the group consisting of the amino acid sequences of references SEQ ID NO: 10 and SEQ ID NO: 11.

In particular the endolysin may comprise a protein sequence consisting of the amino acid sequence of reference SEQ ID NO: 10.

According to a particular embodiment, the endolysin comprises a protein sequence encoded by a nucleic acid sequence comprising at least 80%, in particular at least 90%, more particularly at least 95% sequence identity with a nucleic acid sequence of reference SEQ ID NO: 12. In particular, the endolysin may comprise a protein sequence encoded by a nucleic acid sequence consisting of a nucleic acid sequence of reference SEQ ID NO: 12.

An endolysin comprising a protein sequence encoded by a nucleic acid sequence of reference SEQ ID NO: 12 differs from the Φ2638a bacteriophage endolysin of S. aureus in that the N-terminal M23 endopeptidase domain is substituted with an M23 endopeptidase domain from the lysostaphin of S. simulans.

Endolysins that are suitable for use in the invention may be obtained via any method known to those skilled in the art for producing recombinant proteins. In particular, endolysins according to the invention may be obtained by introducing one or more gene(s) of interest, such as the nucleic acid sequences described previously, into the genome of a host organism via a vector.

In another aspect, a nucleic acid construct comprising at least one of the nucleic acid sequences as defined previously is described. This nucleic acid construct may comprise a first nucleic acid sequence encoding a polypeptide comprising a cell wall binding domain, also possibly comprising a second and third nucleic acid sequence as defined previously. Also described is an expression vector comprising such a nucleic acid construct. Preferably, an expression vector comprises a nucleotide sequence as mentioned previously, which is operatively linked to one or more control sequences, which direct the production or expression of the encoded polypeptide in a cell, a subject or a cell-free expression system. An expression vector may be considered a recombinant expression vector. This vector may consist of a plasmid, cosmid, bacteriophage or virus which is transformed by the introduction of a nucleic acid molecule according to the invention. Such transformation vectors according to the host organism to be transformed are well known to those skilled in the art and widely described in the literature.

Another subject described herein is a process for transforming host organisms by integration of at least one nucleic acid sequence as described, which transformation may be performed via any suitable means known and widely described in the specialized literature, more particularly via the vector described above.

In another aspect, a cell is described, which comprises a nucleic acid construct or an expression vector as defined previously. A cell may be any microbial, prokaryotic or eukaryotic cell, which is suitable for expressing an endolysin that is suitable for use in the invention. In a preferred embodiment, said cell is an E. coli cell. In an even more preferred embodiment, said cell is E. coli CL1blue MRF.

The endolysin obtained may then be purified according to purification methods known in the art such as column chromatography, high performance liquid chromatography, etc. In a particular embodiment, one or more of the protein sequences as defined in the text may comprise a sequence encoding a tag to facilitate the purification of the resulting endolysin. Preferably, said tag is chosen from, but is not limited to, a group consisting of a FLAG tag, a poly(His) tag, an HA tag and an Myc tag. More preferably, said tag is a 6×His tag. Even more preferably, said tag is an N-terminal 6×His tag identical to SEQ ID NO: 13.

Endolysins that are suitable for use according to the invention and also methods for producing them are notably described in patent application WO 2012/150858 A1.

An endolysin, in particular an endolysin derived from a Staphylococcus aureus phage, that is suitable for use according to the invention may be present in the composition in freshly prepared form or in lyophilized form.

In the present text, the term “freshly prepared” is preferably defined as storage for no more than 2 days after production at 1.63 mg/mL in lyophilization buffer (50 mM Tris, 500 mM sucrose, 200 mM mannitol, 0.05% polysorbate 20+50% glycerol) at −20° C. followed by thawing immediately prior to evaluating the lytic activity in an assay as identified herein. The term “lyophilized” refers to an endolysin that has been dehydrated by lyophilization, which consists in freezing the protein and then dehydrating it to remove the water.

After lyophilization, an endolysin in freeze-dried form may undergo a subsequent reconstitution step by addition of water. In one embodiment, the lyophilization and reconstitution may be performed by dialysis against three changes of 300 ml lyophilization buffer (50 mM phosphate or Tris, 500 mM sucrose, 200 mM mannitol, pH 7.4) aliquot and freezing in the gas phase of liquid nitrogen. The lyophilization may be performed under standard conditions, preferably at −40° C. and under vacuum at 75 mTorr for 60 minutes, before increasing the temperature over 5 hours to −10° C. and further increasing over 60 minutes to −10° C. at the same vacuum levels. As a final step, the temperature is preferably increased to 25° C. over 10 hours. The samples are reconstituted by adding water.

A composition according to the invention may comprise a content of endolysin(s) ranging from 0.0001% to 0.1% by weight relative to the total weight of the composition, in particular from 0.0005% to 0.01% by weight relative to the total weight of the composition, more particularly from 0.001% to 0.005% by weight relative to the total weight of the composition.

### Organic Fillers

The composition according to the invention comprises at least one organic filler chosen from an unmodified starch, an N-acylamino acid, salts thereof and mixtures thereof.

### Unmodified Starches

The composition according to the present invention may comprise one or more unmodified starches.

For the purposes of the present invention, the term “unmodified starch” means native starch, or else starch which has not been chemically or physically modified, notably by one or more of the following reactions: pregelatinization, oxidation, crosslinking, esterification, etherification, amidation, heat treatment.

The unmodified starch molecules that may be used in the present invention may originate from any plant source of starch, notably cereals and tubers; more particularly, they may be starches from corn, rice, cassava, barley, potato, wheat, sorghum, pea or oat.

In one particular embodiment, the unmodified starch is chosen from corn starches, rice starches, potato starches and mixtures thereof; preferably, the unmodified starch is chosen from corn or potato starches.

In one particular embodiment, the composition according to the invention is totally free of tapioca starch; more preferentially, the composition according to the invention is totally free of unmodified tapioca starch.

In one particular embodiment, the unmodified starch used in the composition of the present invention is a corn starch such as that sold under the name Beauty-by-Roquette ST005 by the company Roquette.

The starch(es) may be present in the composition according to the invention in a content ranging from 0.1% to 10% by weight, preferably from 0.5% to 5% by weight, better still from 1% to 2.5% by weight, such as 1%, 1.5% or 2% by weight, relative to the total weight of the composition.

### N-Acylamino Acids and Salts Thereof

The composition according to the present invention may comprise one or more N-acylamino acids, salts thereof and mixtures thereof.

The term “salt” of an N-acylamino acid according to the invention means a salt formed by an inorganic or organic acid or an inorganic or organic base.

As examples of acid salts, mention may be made of the sulfate, citrate, acetate, oxalate, chloride, bromide, iodide, nitrate, bisulfate, phosphate, isonicotinate, lactate, salicylate, tartrate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and aspartate salts.

As examples of base salts, mention may be made of hydroxides of alkali metals such as sodium, potassium and lithium; hydroxides of alkaline-earth metals such as calcium and magnesium; hydroxides of other metals such as aluminium and zinc; aqueous ammonia and organic amines such as unsubstituted or hydroxy-substituted mono-, di- or trialkylamines; dicyclohexylamines; tributylamines; pyridine; N-methyl-N-ethylamine; diethylamine; triethylamine; mono-, bis- or tris(2-hydroxyalkylamines) such as mono-, bis- or tris(2-hydroxyethyl)amine, 2-hydroxy-tert-butylamine or tris(hydroxymethyl)methylamine, N,N-dialkyl-N-(hydroxyalkyl)amines, such as N,N-dimethyl-N-(2-hydroxyethyl)amine; N-methyl-D-glucamine; and amino acids such as arginine and lysine.

N-Acylamino acids that are suitable as organic fillers according to the invention comprise at least one acyl group containing from 8 to 22 carbon atoms, in particular a 2-ethylhexanoyl, caproyl, lauroyl, myristoyl, palmitoyl, stearoyl or cocoyl group, preferably lauroyl.

The amino acid may be, for example, lysine, glutamic acid or alanine, preferably lysine.

In particular, the amino acid may be of D or L configuration, more particularly L.

According to a particular embodiment, the C8-C22 N-acylamino acids are chosen from lauroyl lysine, salts thereof and mixtures thereof, more particularly N-lauroyl-L-lysine, salts thereof and mixtures thereof.

N-Lauroyl-L-lysine is notably sold under the name Amihope LL® by the company Ajinomoto.

The N-acylamino acid(s) and the salts thereof may be present in the composition according to the invention in a content ranging from 0.1% to 15% by weight, preferably from 1% to 10% by weight, better still from 2% to 4% by weight, such as 3% by weight, relative to the total weight of the composition.

The organic filler(s) may be present in the composition according to the invention in a total content ranging from 0.1% to 15% by weight, preferably from 0.5% to 10% by weight, better still from 1% to 4% by weight relative to the total weight of the composition.

A composition according to the invention may comprise one or more endolysins according to the invention and one or more organic fillers according to the invention in an endolysin(s)/organic filler(s) mass ratio of between 0.0001 and 0.04, preferably between 0.0002 and 0.004 and better still between 0.0006 and 0.002.

A composition according to the invention may comprise at least water, and optionally a water-miscible organic solvent.

In particular, a composition according to the invention may include an amount of water of at least 10% by weight relative to the total weight of the composition, in particular an amount of water ranging from 10% to 98% by weight, more particularly from 20% to 95% by weight, notably from 30% to 90% by weight and more particularly from 35% to 85% by weight relative to the total weight of the composition.

The water may be sterile demineralized water and/or floral water and/or natural spring or mineral water.

In the present invention, the term “water-miscible organic solvent” denotes an organic compound which is liquid at room temperature and whose miscibility in water is greater than 50% by weight at 25° C. and atmospheric pressure.

The water-miscible organic solvents that may be used in a composition of the invention may also be volatile.

Among the water-miscible organic solvents that may be used in a composition of the invention, mention may be made, for example, of lower monoalcohols containing from 2 to 5 carbon atoms, such as ethanol and isopropanol, polyols such as propylene glycol and glycerol.

According to one embodiment, a composition according to the invention may be free of lower monoalcohols containing 2 to 5 carbon atoms.

A composition according to the invention may comprise less than 2% by weight of ethanol, preferably less than 1% by weight, more particularly less than 0.5% by weight, relative to the total weight of the composition, in particular less than 0.1% by weight of ethanol, and preferably may be free of ethanol.

### Additional Ingredients

In addition to the abovementioned compounds, a composition according to the invention may of course comprise one or more additional ingredients.

Needless to say, a person skilled in the art will take care to select one or more additional ingredients such that the advantageous properties of the endolysin(s) of the invention are not, or are not substantially, adversely affected.

The additional ingredients are present in the compositions in a content which is usual for each of them in a cosmetic composition, in particular in a content which is usual for each of them and which enables them to retain their cosmetic properties, more particularly in a content which is usual for each of them and which enables them, when they are each the only ingredient of a composition according to the invention having this property, to retain their cosmetic property.

Thus, a composition according to the invention may comprise one or more of the following ingredients, chosen from: surfactants; fatty substances; colorants; preserving agents; fragrances; pH adjusters such as organic acids, for instance citric acid; antioxidants; hydrophilic gelling agents such as hydroxypropylmethylcellulose; amino acids such as arginine; carbohydrates; chelating agents; sugar alcohols; cosmetic active agents; and mixtures thereof.

Needless to say, a person skilled in the art will take care to select this or these optional additional ingredient(s), and/or the amount thereof, such that the advantageous properties of a composition according to the invention are not, or are not substantially, adversely affected by the envisioned addition.

The additional ingredient(s) different from those listed below may be present in the composition according to the invention in a concentration of between 0.001% and 20% by weight, in particular from 0.01% to 10% by weight, more particularly between 0.1% and 5% by weight relative to the total weight of the composition.

According to a particular embodiment, a composition according to the invention may comprise at least one additional ingredient chosen from an oil, an aromatic alcohol of formula (I), a nonionic surfactant, and mixtures thereof.

### Oil

A composition according to the invention may comprise at least one oil.

For the purposes of the present invention, the term “oil” denotes a water-immiscible compound which is liquid at 25° C. and atmospheric pressure (1.013×10⁵ Pa).

The term “immiscible” means that the mixing of the same amount of water and oil, after stirring, does not result in a stable solution comprising only a single phase, under the abovementioned temperature and pressure conditions. Observation is performed by eye or using a phase-contrast microscope, if necessary, on 100 g of mixture obtained after sufficient stirring with a Rayneri blender to produce a vortex within the mixture (as a guide, 200 to 1000 rpm), the resulting mixture being left to stand, in a closed flask, for 24 hours at room temperature before observation.

The term “hydrocarbon-based oil” refers to an oil mainly containing carbon and hydrogen atoms and possibly one or more functions chosen from hydroxyl, ester, ether and carboxylic functions. A hydrocarbon-based oil thus consequently does not comprise any silicon or fluorine atoms.

The term “silicone oil” refers to an oil comprising at least one silicon atom, and notably at least one Si—O group, and more particularly an organopolysiloxane.

The term “fluoro oil” denotes an oil comprising at least one fluorine atom.

The term “apolar hydrocarbon-based oil” means a hydrocarbon-based oil comprising only carbon and hydrogen atoms, which is in particular non-aromatic (also called a hydrocarbon). The term “polar hydrocarbon-based oil” denotes hydrocarbon-based oils mainly comprising carbon and hydrogen atoms and one or more functions chosen from hydroxyl, ester, ether and carboxylic functions.

In particular, the composition according to the invention may comprise at least one oil chosen from volatile and non-volatile oils, in particular with the exception of liquid paraffins.

### Volatile Oils

The term “volatile oil” means an oil (or non-aqueous medium) that can evaporate on contact with the skin in less than one hour, at room temperature and at atmospheric pressure. The volatile oil is a volatile cosmetic oil, which is liquid at room temperature, notably having a non-zero vapour pressure, at room temperature and at atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

According to a particular embodiment of the invention, the volatile oils are such that the flash points are less than 120° C. and the vapour pressure is less than 5 Pa, more particularly the flash point is less than 90° C. and the vapour pressure is greater than 1 Pa, even more particularly the flash point is less than or equal to 60° C. and the vapour pressure is greater than 5 Pa, and even more particularly the flash point is less than 60° C. and the vapour pressure is greater than 100 Pa.

The volatile oil(s) may be chosen from volatile hydrocarbon-based oils such as:
hydrocarbon-based oils containing from 8 to 16 carbon atoms, and notably:
a) branched C8-C16 alkanes such as isoalkanes (also known as isoparaffins) such as C8-C9 Isoparaffin, C13-C16 Isoparaffin, isododecane, isodecane, isohexadecane, and for example oils sold under the trade names Isopar or Permethyl, alone or as mixtures, in particular isododecane (also known as 2,2,4,4,6-pentamethylheptane), for example sold by Ineos, more particularly isododecane;
b) linear C6-C16 alkanes, alone or as mixtures, for example such as hexane, decane, undecane, tridecane, or n-dodecane (C12) and n-tetradecane (C14) sold by Sasol under the respective references Parafol 12-97 and Parafol 14-97, the undecane-tridecane mixture, the mixtures of n-undecane (C11) and n-tridecane (C13) obtained in Examples 1 and 2 of patent application WO 2008/155059 by the company Cognis, and mixtures thereof, and also mixtures of n-undecane (C11) and n-tridecane (C13) Cetiol Ultimate® from the company BASF;
c) volatile C5-C12 cyclic, non-aromatic alkanes;
short-chain esters containing 3 to 8 carbon atoms in total, such as methyl acetate, ethyl acetate, propyl acetate, n-butyl acetate or isobutyl acetate, for example sold by Solvay, Dow or Oxea;
volatile carbonate hydrocarbon-based oils of structure R′1-O—C(O)—O—R′2 in which R′1 and R′2, which may be identical or different, independently denote a linear, branched or cyclic C4-C8 alkyl group, in particular a linear C4-C8 alkyl group. It may be preferable for R1 and R2 to be identical. In particular R′1 and R′2 denote a linear butyl alkyl radical or a pentyl group. Advantageously, the ether oil is chosen from dibutyl carbonate or dipentyl carbonate;
volatile ether oils of formula R1-O—R2 in which R¹ and R2, which may be identical or different, independently denote a linear, branched or cyclic C4-C8 alkyl group, in particular a linear or branched C4-C8 alkyl group. It is preferable for R1 and R² to be identical. Linear alkyl groups that may be mentioned include a butyl group and a pentyl group. Branched alkyl groups that may be mentioned include a 1-methylpropyl group, a 2-methylpropyl group, a t-butyl group and a 1,1-dimethylpropyl group.

In particular, the hydrocarbon-based volatile oil(s) are chosen from C8-C16 alkanes, in particular linear alkanes, and more particularly are chosen from C9-C12 alkanes, even more particularly are chosen from a mixture of C9-C12 alkanes such as Vegelight Silk® sold by BioSynthIs.

The volatile oil(s) may be chosen from volatile silicone oils such as:
silicone oils comprising, in particular, from 2 to 7 silicon atoms, these silicone oils optionally including alkyl or alkoxy groups containing from 1 to 10 carbon atoms. As volatile silicone oils that may be used in the invention, mention may notably be made of dimethicones with viscosities of 5 and 6 cSt, cyclopentadimethylsiloxane, dodecamethylpentasiloxane, cyclohexadimethylsiloxane, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof. Mention may notably be made of dodecamethylpentasiloxane, such as DM-Fluid-2cs sold by Shin-Etsu, or cyclohexadimethylsiloxane, such as the reference Xiameter PMX-0246 Cyclohexasiloxane sold by Dow Chemical.

### Non-Volatile Oils

The term “non-volatile oil” means an oil whose vapour pressure at 25° C. and atmospheric pressure is non-zero and is less than 2.66 Pa and more particularly less than 0.13 Pa. By way of example, the vapour pressure may be measured according to the static method or via the effusion method by isothermal thermogravimetry, depending on the vapour pressure of the oil (standard OCDE 104).

The non-volatile oil(s) may be of natural or synthetic origin, in particular natural.

Among the non-volatile oils, mention may be made of:
non-volatile fluoro oils, which may notably be chosen from among fluorinated polyethers, and also from the fluorosilicone oils and the fluoro silicones as described in EP-A-847752;
non-volatile silicone oils, which may notably be chosen from the non-volatile silicones having the following INCI names: dimethicone, dimethiconol, trimethyl pentaphenyl trisiloxane, tetramethyl tetraphenyl trisiloxane, diphenyl dimethicone, trimethylsiloxyphenyl dimethicone, phenyl trimethicone, diphenylsiloxyphenyl trimethicone; and also mixtures thereof.

These products are notably sold under the names PH-1555 HRI Cosmetic Fluid (trimethyl pentaphenyl trisiloxane) and Dow Corning 556 Cosmetic Grade Fluid (phenyl trimethicone) by Dow Corning; diphenyl dimethicones such as the products KF-54, KF54HV, KF-50-300CS, KF-53 d and KF-50-100CS or Diphenylsiloxy Phenyl Trimethicone KF56 A sold by Shin-Etsu; the products Belsil PDM 1000 and Belsil PDM 20 sold by Wacker Chemie (trimethylsiloxy phenyl dimethicone), alone or as mixtures;
non-volatile apolar hydrocarbon-based oils, which may notably be chosen from linear or branched compounds of mineral or synthetic origin, for instance: i) squalane such as the reference Neossance Squalane sold by Amyris, isoeicosane, ii) mixtures of linear, saturated hydrocarbons, particularly C14-C30 and more particularly C15-C28 hydrocarbons, such as mixtures whose INCI names are, for example, the following: (C15-C19)Alkane, (C18-C21)Alkane, (C21-C28)Alkane, for instance the products Gemseal 40, Gemseal 60 and Gemseal 120 sold by Total, Emogreen L19 sold by SEPPIC, Emogreen L15 sold by SEPPIC, iii) hydrogenated or non-hydrogenated polybutenes, for instance the products of the Indopol range sold by Ineos Oligomers, products having the INCI name Hydrogenated Polyisobutene; iv) hydrogenated or non-hydrogenated polyisobutenes, in particular hydrogenated, for instance the non-volatile compounds of the Parleam® range sold by the company Nippon Oil Fats, v) hydrogenated or non-hydrogenated polydecenes, for instance the non-volatile compounds of the Puresyn® range sold by the company ExxonMobil), vi) decene/butene copolymers, butene/isobutene copolymers, and vii) mixtures thereof;
non-volatile polar hydrocarbon-based oils, which may be chosen from:
i) saturated, unsaturated, linear or branched C10-C26 fatty alcohols, which are liquid at room temperature (25° C.), in particular monoalcohols. In particular, the C10-C26 alcohols are fatty alcohols, which are in particular branched when they comprise at least 16 carbon atoms; in particular, the fatty alcohol comprises from 10 to 24 carbon atoms, and more particularly from 12 to 22 carbon atoms, notably such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
ii) triglycerides consisting of fatty acid esters of glycerol, in particular the fatty acids of which may have chain lengths ranging from C4 to C36, and notably from C18 to C36, these oils possibly being linear or branched, and saturated or unsaturated; by way of example, mention may notably be made of heptanoic or octanoic triglycerides, caprylic/capric acid triglycerides, plant oils such as wheat germ oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon-based esters of formula R—C(O)—OR′ in which R—C(O)— represents a carboxylic acid residue including from 2 to 40 carbon atoms and R′ represents a hydrocarbon-based chain containing from 1 to 40 carbon atoms, aliphatic hydrocarbon-based esters of alkylene glycol, in particular ethylene glycol or propylene glycol, the total number of carbon atoms advantageously being at least 10. As examples of such esters, mention may be made of isoamyl laurate, cetostearyl octanoate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate, cocoyl caprylate/caprate, tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, such as isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate; and mixtures thereof;
iv) hydroxylated esters such as polyglyceryl-2 triisostearate;
v) aromatic esters such as tridecyl trimellitate, C12-C15 alcohol benzoate, the 2-phenylethyl ester of benzoic acid, and butyloctyl salicylate;
vi) linear fatty acid esters with a total carbon number ranging from 35 to 70, for instance pentaerythrityl tetrapelargonate;
vii) esters of C24-C28 branched fatty acids or fatty alcohols such as triisoarachidyl citrate, pentaerythrityl tetraisononanoate, glyceryl triisostearate, glyceryl tris(2-decyltetradecanoate), pentaerythrityl tetraisostearate, polyglyceryl-2 tetraisostearate or pentaerythrityl tetrakis(2-decyltetradecanoate);
viii) the polyesters obtained by condensation of dimer and/or trimer of unsaturated fatty acid and of diol, such as those with the INCI name Dilinoleic Acid/Butanediol Copolymer or Dilinoleic Acid/Propanediol Copolymer; the polyesters obtained by condensation of fatty acid dimer and of diol dimer, such as dimer dilinoleyl dimer dilinoleate;
ix) synthetic ethers of formula R1-O—R2 in which R1 and R2, which may be identical or different, independently denote a linear, branched or cyclic C6-C24 alkyl group, in particular a C6-C18 alkyl group, and more particularly a C8-C12 alkyl group. It may be preferable for R1 and R2 to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. As cyclic alkyl groups, mention may be made of a cyclohexyl group, a 3-methylcyclohexyl group and a 3,3,5-trimethylcyclohexyl group, dilauryl ether, diisostearyl ether, dioctyl ether, nonylphenyl ether, dodecyl dimethylbutyl ether, cetyl dimethylbutyl ether, cetyl isobutyl ether and mixtures thereof. Among the non-volatile ether oils, mention may be made of dicaprylyl ether, such as the reference Cetiol OE sold by BASF;
x) carbonates of formula R8-O—C(O)—O—R9, with R8 and R9, which may be identical or different, representing a linear or branched C4 to C12 and in particular C6 to C10 alkyl chain; the carbonate oils may be dicaprylyl carbonate (or dioctyl carbonate), sold under the name Cetiol CC® by the company BASF, bis(2-ethylhexyl) carbonate, sold under the name Tegosoft DEC® by the company Evonik, dipropylheptyl carbonate (Cetiol 4 AII from BASF), dibutyl carbonate, dineopentyl carbonate, dipentyl carbonate, dineoheptyl carbonate, diheptyl carbonate, diisononyl carbonate or dinonyl carbonate, and more particularly dioctyl carbonate;
xi) vinylpyrrolidone copolymers such as vinylpyrrolidone/1-hexadecene copolymer (INCI name);
xii) higher C6-C26 fatty acids which are liquid at room temperature (25° C.), such as oleic acid, linoleic acid, linolenic acid or isostearic acid; and
xiii) mixtures thereof.

According to a particular embodiment, the composition comprises at least one oil chosen from volatile C8-C16 alkane hydrocarbon-based oils, non-volatile silicone oils, apolar non-volatile hydrocarbon-based oils with the exception of liquid paraffins, polar non-volatile hydrocarbon-based oils as defined previously, and mixtures thereof more particularly chosen from polar non-volatile hydrocarbon-based oils and mixtures thereof.

According to a particular embodiment, the composition comprises at least one oil chosen from polar hydrocarbon-based non-volatile oils; more particularly, the composition according to the invention comprises at least one oil chosen from polar hydrocarbon-based non-volatile oils and does not comprise any apolar hydrocarbon-based non-volatile oils; even more particularly, the composition according to the invention comprises at least one oil chosen from polar hydrocarbon-based non-volatile oils and does not comprise any apolar hydrocarbon-based non-volatile oils, fluorinated non-volatile oils, silicone-based non-volatile oils or volatile oils.

According to a particular embodiment, the polar hydrocarbon-based non-volatile oils are chosen from:
i) saturated, unsaturated, linear or branched C10-C26 fatty alcohols, which are liquid at room temperature (25° C.), in particular monoalcohols. In particular, the C10-C26 alcohols are fatty alcohols, which are in particular branched when they comprise at least 16 carbon atoms; more particularly, the fatty alcohol comprises from 10 to 24 carbon atoms, and even more particularly from 12 to 22 carbon atoms, notably such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
ii) triglycerides consisting of fatty acid esters of glycerol, in particular the fatty acids of which may have chain lengths ranging from C4 to C36, and notably from C18 to C36, these oils possibly being linear or branched, and saturated or unsaturated; by way of example, mention may notably be made of heptanoic or octanoic triglycerides, caprylic/capric acid triglycerides, plant oils such as wheat germ oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon-based esters of formula R—C(O)—OR′ in which R—C(O)— represents a carboxylic acid residue including from 2 to 40 carbon atoms and R′ represents a hydrocarbon-based chain containing from 1 to 40 carbon atoms, aliphatic hydrocarbon-based esters of alkylene glycol, in particular ethylene glycol or propylene glycol, the total number of carbon atoms advantageously being at least 10. As examples of such esters, mention may be made of isoamyl laurate, cetostearyl octanoate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate, cocoyl caprylate/caprate, tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, such as isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate; and mixtures thereof;
ix) synthetic ethers of formula R1-O—R2 in which R1 and R2, which may be identical or different, independently denote a linear, branched or cyclic C6-C24 alkyl group, in particular a C6-C18 alkyl group, and more particularly a C8-C12 alkyl group. It may be preferable for R1 and R2 to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. As cyclic alkyl groups, mention may be made of a cyclohexyl group, a 3-methylcyclohexyl group and a 3,3,5-trimethylcyclohexyl group, dilauryl ether, diisostearyl ether, dioctyl ether, nonylphenyl ether, dodecyl dimethylbutyl ether, cetyl dimethylbutyl ether, cetyl isobutyl ether and mixtures thereof. Among the non-volatile ether oils, mention may be made of dicaprylyl ether, such as the reference Cetiol OE sold by BASF;
x) carbonates of formula R8-O—C(O)—O—R9, with R8 and R9, which may be identical or different, representing a linear or branched C4 to C12 and in particular C6 to C10 alkyl chain; the carbonate oils may be dicaprylyl carbonate (or dioctyl carbonate), sold under the name Cetiol CC® by the company BASF, bis(2-ethylhexyl) carbonate, sold under the name Tegosoft DEC® by the company Evonik, dipropylheptyl carbonate (Cetiol 4 AII from BASF), dibutyl carbonate, dineopentyl carbonate, dipentyl carbonate, dineoheptyl carbonate, diheptyl carbonate, diisononyl carbonate or dinonyl carbonate, and more particularly dioctyl carbonate;
More particularly, the polar hydrocarbon-based non-volatile oils are chosen from:
ii) triglycerides consisting of fatty acid esters of glycerol, in particular the fatty acids of which may have chain lengths ranging from C4 to C36, and notably from C18 to C36, these oils possibly being linear or branched, and saturated or unsaturated; by way of example, mention may notably be made of heptanoic or octanoic triglycerides, caprylic/capric acid triglycerides, plant oils such as wheat germ oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof.

According to a particular embodiment, the composition comprises at least one oil chosen from the group consisting of:
non-volatile polar hydrocarbon-based oils of the triglyceride type constituted by fatty acid esters of glycerol, in particular whose fatty acids may have chain lengths ranging from C4 to C36, and notably from C18 to C36, these oils possibly being linear or branched, saturated or unsaturated, chosen from soybean oil, jojoba seed oil, shea butter olein, capric and caprylic acid triglycerides, and mixtures thereof,
volatile C8-C16 alkane oils, such as C9-C12 alkanes and isoparaffin,
apolar non-volatile oils such as squalane,
non-volatile polar hydrocarbon-based oils of the synthetic ether type of formula R1-O—R2, in which R1 and R2, which may be identical or different, independently denote a linear, branched or cyclic C6-C24 alkyl group, in particular a C6-C18 alkyl group, and more particularly a C8-C12 alkyl group, such as dicaprylyl ether,
non-volatile silicone oils such as dimethicone,
polar hydrocarbon-based non-volatile oils of the saturated, unsaturated, linear or branched C10-C26 fatty alcohol type, which are liquid at room temperature (25° C.), such as octyldodecanol,
non-volatile polar hydrocarbon-based oils such as the carbonates of formula R8-O—C(O)—O—R9, in which R8 and R9, which may be identical or different, represent a linear or branched C4 to C12, in particular C6 to C10, alkyl chain, such as dicaprylyl carbonate, and
mixtures thereof.

According to a particular embodiment, the oil is chosen from the group consisting of octyldodecanol, soybean oil, shea butter olein, dicaprylyl carbonate, dimethicone, jojoba oil, isoparaffin, isononyl isonanoate, caprylic/capric acid triglycerides, C9-C12 alkanes, squalane, dicaprylyl ether, and mixtures thereof.

According to a particular embodiment, the oil is chosen from the group consisting of soybean oil, shea butter olein, jojoba oil, isononyl isonanoate, caprylic/capric acid triglycerides, and mixtures thereof.

According to a particular embodiment, the oil is different from a liquid paraffin.

According to a particular embodiment, the composition is free of liquid paraffins, i.e. the composition comprises 0% liquid paraffins.

The oils according to the invention may advantageously be present in a composition according to the invention in a content customary for a cosmetic composition, in particular in a customary content allowing them to play their cosmetic role in a composition of the invention, in particular in a cosmetic composition according to the invention, more particularly in a customary content allowing them to play their cosmetic role when they are the only ones to play this role in a composition according to the invention.

The oil may play various cosmetic roles, such as that of a consistency factor, holding an emulsion in the cold, or obtaining the smooth appearance of the composition, notably in the case of an emulsion. It may also contribute towards facilitating the spreading and glidance of the composition on the skin, and thus its penetration. Finally, the oil may act on the skin through its occlusive effect, its lubricating effect (to the touch) or its emollient/hydrating effect.

A composition according to the invention may comprise a total oil content ranging from 1% to 80% by weight relative to the total weight of the composition, in particular from 2% to 60% by weight relative to the total weight of the composition, more particularly from 5% to 40% by weight, even more particularly from 10% to 30% by weight relative to the total weight of the composition.

The term “total oil content” means the sum of the contents of each of the previously mentioned oils present in the composition, or, when only one of these oils is present in the composition, it means the content of this oil.

### Aromatic Alcohols

A composition according to the invention may also comprise at least one aromatic alcohol of formula (I), a salt thereof, notably a salt of an organic or mineral base, an optical isomer thereof, a geometrical isomer thereof or a solvate thereof, such as hydrate:

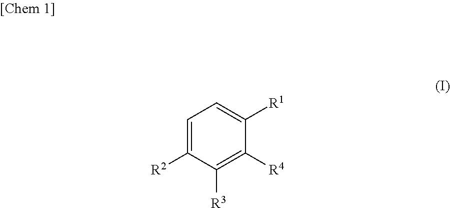

in which:
   R1 represents a group chosen from the group consisting of:
a) linear or branched hydroxy (C1-C4)alkyl, in particular a hydroxy (C1-C2)alkyl group,
b) a group chosen from —OR⁵, —C(O)R⁶, —C(O)OR⁷ and —(CH₂)ₙ—C(H)(R⁸)—C(O)R⁹, with:
R⁵ representing a linear or branched (C₃-C₄)alkyl group optionally substituted with one or more hydroxyl (OH) groups,
R⁶ representing a hydrogen atom or a linear or branched (C₁-C₄)alkyl, phenyl or benzyl group, optionally substituted with one or more hydroxyl groups,
R⁷ representing a linear or branched (C₁-C₄)alkyl, phenyl or benzyl group, optionally substituted with one or more hydroxyl groups,
R⁸ representing a hydrogen atom or a linear or branched (C₁-C₄)alkyl group such as methyl or ethyl, in particular R⁸ representing a hydrogen atom;
R⁹ representing a hydrogen atom, or a linear or branched, in particular linear, (C₁-C₁₂)alkyl group optionally substituted with one or more hydroxyl groups, or a linear or branched, in particular linear, (C₂-C₁₂)alkenyl group optionally substituted with one or more hydroxyl groups,
n is 0, 1 or 2, in particular n is 1,
   R² represents a hydrogen atom; a halogen atom, in particular a chlorine atom; or a hydroxyl group; and
   R³ represents a hydrogen atom or a group chosen from hydroxyl and linear or branched (C₁-C₆)alkoxy, in particular (C₁-C₄)alkoxy such as methoxy —OCH₃ or ethoxy —OC₂H₅; in particular, R³ represents a hydrogen atom or an ethoxy group; and
   R⁴ is a hydrogen atom or a hydroxyl group;
it being understood that at least one of the groups R¹, R², R³ or R⁴ bears or represents a hydroxyl group.

More particularly, the aromatic alcohol(s) of formula (I) of the invention are such that:
when R¹ represents a group —C(O)OR⁷, then R² is an —OH group;
when R¹ represents a group —C(O)R⁶, then at least one from among R², R³ and R⁴ is an —OH group;
when R¹ represents a group —(CH₂)ₙ—C(H)(R⁸)—C(O)R⁹, then R² is an —OH group.

In particular, the aromatic alcohol may have the formula (II) below:

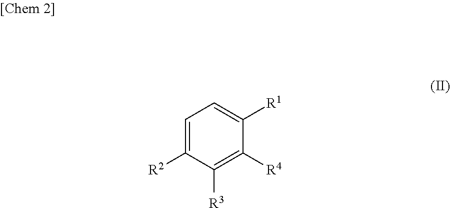

in which:
R¹ is chosen from the group consisting of:
i) a linear or branched hydroxy (C₁-C₄)alkyl, in particular a hydroxy (C₁-C₂)alkyl group such as hydroxymethyl or hydroxyethyl,
ii) a group —OR⁵ with R⁵ representing a (C₃-C₄) hydroxyalkyl group, notably —OR⁵ representing —O—CH₂—CH(OH)—CH₂OH,
iii) a group —C(O)R⁶ with R⁶ representing a linear or branched (C₁-C₄)alkyl group, notably-C(O)R⁶ representing —C(O)—CH₃,
iv) a group —C(O)OR⁷ with R⁷ representing a linear or branched (C₁-C₄)alkyl group, notably R⁷ representing a methyl, ethyl, propyl, isopropyl, butyl, isobutyl or benzyl group, and
v) a group —CH₂—CH₂—C(O)R⁹ with R⁹ representing a linear or branched (C₁-C₆)alkyl group, in particular —CH₂—CH₂—C(O)R⁹ representing a —CH₂—CH₂—C(O) CH₃ group;
R² is chosen from the group consisting of a hydrogen atom; a halogen atom, in particular a chlorine atom; and a hydroxyl group;
R³ is a hydrogen atom or a methoxy or ethoxy group; and
R⁴ is a hydrogen atom or a hydroxyl group;
it being understood that at least one of the groups R¹, R² or R⁴ bears or represents a hydroxyl group.

Such compounds notably act as preserving agents, in particular in cosmetic compositions. Some also act as fragrances, notably in cosmetic compositions.

The term “organic or mineral base salt” means salts of bases or alkaline agents as defined below. As examples of base salts, mention may be made of alkali metal hydroxides such as sodium, potassium and lithium hydroxides; alkaline-earth metal hydroxides such as calcium and magnesium hydroxides; hydroxides of other metals, such as aluminium and zinc hydroxides; ammonia and organic amines such as unsubstituted or hydroxy-substituted mono-, di- or tri-alkylamines; dicyclohexylamines; tributylamines; pyridine; N-methyl-N-ethylamine; diethylamine; triethylamine; mono-, bis- or tris-(2-hydroxyalkylamines) such as mono-, bis- or tris-(2-hydroxyethyl)amine, 2-hydroxy-tert-butylamine, tris(hydroxymethyl)methylamine; N,N-dialkyl-N-(hydroxyalkyl)amines, such as N,N-dimethyl-N-(2-hydroxyethyl)amine; N-methyl-D-glucamine; and amino acids such as arginine and lysine.

For the purposes of the present invention, the term “alkyl group” or “alkyl radical” means a saturated, linear or branched, substituted or unsubstituted monovalent hydrocarbon-based radical, in particular methyl, ethyl, propyl, isopropyl, butyl or tert-butyl.

The term “hydroxyalkyl group” means a saturated, linear or branched hydrocarbon-based group comprising at least one-OH group. A hydroxy (C₁-C₄)alkyl group is a saturated, linear or branched C₁-C₄ hydrocarbon-based radical comprising at least one-OH group, in particular comprising a single-OH group. A hydroxy (C₁-C₂)alkyl group is a saturated, linear or branched C₁-C₂ hydrocarbon-based radical comprising at least one-OH group, in particular comprising a single-OH group.

According to a particular embodiment, the hydroxy (C₁-C₄)alkyl group is chosen from the group consisting of hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl. In particular, the hydroxyl(C₁-C₄)alkyl group is hydroxymethyl or 2-hydroxyethyl.

The term “halogen atom” means one of the chemical elements of Group 17 of the Periodic Table of the Elements, namely fluorine, chlorine, bromine or iodine. In particular, the halogen atom is a chlorine atom.

According to a particular embodiment, R¹ is chosen from the group constituted by (C₁-C₂) hydroxyalkyl, 2-hydroxyethyl, hydroxymethyl, an —O—CH₂—CH(OH)—CH₂OH group, a —CH₂—CH₂—C(O)—CH₃ group and a —C(O)—OCH₃ group,
R² is chosen from the group constituted by a hydrogen atom, a halogen atom, in particular a chlorine atom, and a hydroxyl group,
R³ is chosen from the group constituted by a hydrogen atom and an —O—C₂—H₅ group, and
R⁴ is a hydrogen atom.

According to one embodiment, R¹ represents a hydroxy (C₁-C₄)alkyl group, more particularly a hydroxy (C₁-C₂)alkyl group, R² represents a hydrogen atom, R³ represents a hydrogen atom and R⁴ represents a hydrogen atom.

According to a particular embodiment, R¹ represents 2-hydroxyethyl, R² represents a hydrogen atom, R³ represents a hydrogen atom and R⁴ represents a hydrogen atom.

According to a particular embodiment, R¹ represents a hydroxymethyl, R² represents a hydrogen atom, R³ represents a hydrogen atom and R⁴ represents a hydrogen atom.

According to a particular embodiment, R¹ represents a group —OR⁵ with R⁵ representing a linear or branched (C₃-C₄)alkyl group substituted with one or more hydroxyl groups, R² represents a halogen atom, R³ represents a hydrogen atom and R⁴ represents a hydrogen atom. More particularly, R¹ represents a group —OR⁵ with R⁵ representing a linear (C₃-C₄)alkyl group substituted with two hydroxyl groups, R² represents a halogen atom, R³ represents a hydrogen atom and R⁴ represents a hydrogen atom.

According to a particular embodiment, R¹ represents an —O—CH₂—CH(OH)—CH₂OH group, R² represents a chlorine atom, R³ represents a hydrogen atom and R⁴ represents a hydrogen atom.

According to a particular embodiment, R¹ represents a group —(CH₂)ₙ—C(H)(R)⁸—C(O)R⁹, with R⁸ representing a hydrogen atom or a methyl or ethyl group, and R⁹ representing a linear (C₁-C₁₂)alkyl group optionally substituted with a hydroxyl group or a (C₂-C₁₂)alkenyl group optionally substituted with a hydroxyl group, and n is as defined previously, in particular n is 1, R² represents a hydroxyl group, R³ represents an —OCH₃ or —OCH₅ group and R⁴ represents a hydrogen atom. More particularly, R¹ represents a group —CH₂—CH₂—C(O)R⁹, R⁹ representing a linear (C₁-C₁₂)alkyl group, in particular a linear (C₁-C₆)alkyl such as methyl, R² represents a hydroxyl group, R³ represents an —OC₂H₅ group and R⁴ represents a hydrogen atom.

According to a particular embodiment, R¹ represents a group —C(O)OR⁷, with R⁷ representing a linear or branched (C₁-C₄)alkyl, phenyl or benzyl group, optionally substituted with one or more hydroxyl groups, R² represents a hydroxyl group, R³ represents a hydrogen atom and R⁴ represents a hydrogen atom.

According to a particular embodiment, R¹ represents a —C(O)—OCH₃ group, R² represents a hydroxyl group, R³ represents a hydrogen atom and R⁴ represents a hydrogen atom.

According to a particular embodiment, the aromatic alcohol of formula (I) or (II) is chosen from the group consisting of phenylethyl alcohol; benzyl alcohol; chlorphenesin (also known as 3-(4-chlorophenoxy)-1,2-propanediol); zingerone; ethylzingerone (also known as 4-(3-ethoxy-4-hydroxyphenyl) butan-2-one); vanillin; parabens, in particular (C₁-C₆)alkylparabens or arylparabens, in particular methylparaben, ethylparaben, propylparaben, isopropylparaben, butylparaben, isobutylparaben or benzylparaben; salts thereof and mixtures thereof.

According to a particular embodiment, the aromatic alcohol of formula (I) or (II) is chosen from the group consisting of phenylethyl alcohol; benzyl alcohol; chlorphenesin (also known as 3-(4-chlorophenoxy)-1,2-propanediol); zingerone; ethylzingerone (also known as 4-(3-ethoxy-4-hydroxyphenyl) butan-2-one); parabens, in particular methylparaben; salts thereof and mixtures thereof.

The composition according to the invention comprises in particular a total content of aromatic alcohol(s) of formula (I) or (II) ranging from 0.01% to 3% by weight relative to the total weight of the composition, in particular from 0.05% to 1.5% by weight, and more particularly from 0.1% to 1.0% by weight, relative to the total weight of the composition. The term “total content of aromatic alcohol(s) of formula (I) or (II)” means the sum of the contents of each of the aromatic alcohol(s) of formula (I) or (II) present in the composition, or, when only one aromatic alcohol of formula (I) or (II) is present in the composition, means the content of this aromatic alcohol of formula (I) or (II).

### Nonionic Surfactants

The composition according to the invention may also comprise at least one nonionic surfactant, in particular chosen from nonionic surfactants comprising one or more carbohydrate residues, nonionic surfactants of the C₆-C₃₀ fatty acid alkanolamide type.

For the purposes of the present invention, the term “surfactant” means any compound which modifies the surface tension between two surfaces. Surfactant compounds are amphiphilic molecules, i.e. they have two parts of different polarity, one lipophilic (fat-retaining) and apolar, the other hydrophilic (water-miscible) and polar. They thus allow two immiscible phases to be dissolved, by interacting with the apolar (i.e. lipophilic and thus hydrophobic) phase, via its hydrophobic part; whereas with the other phase, which is polar, it will interact via its hydrophilic part.

The term “nonionic surfactant” means a surfactant which has no net charge (i.e. it does not become ionized in water).

### Nonionic Surfactant Comprising One or More Carbohydrate Residues.

The composition according to the invention may also comprise at least one nonionic surfactant comprising one or more carbohydrate residues.

For the purposes of the present invention, the term “carbohydrate” means a sugar- or carbohydrate-type compound, corresponding to a molecule essentially composed of carbon, hydrogen and oxygen atoms.

According to a particular embodiment, the carbohydrate residue(s) are monosaccharides including 5 to 6 carbon atoms, more particularly chosen from glucose, fructose, xylose or galactose, even more particularly glucose.

According to a particular embodiment, the nonionic surfactant(s) comprising one or more carbohydrate residues are chosen from:
(i) fatty acid esters of sugar(s) such as fatty acid (poly)glyceryl esters of glucose or alkylglucose with a linear or branched, saturated or unsaturated, C6-C22, in particular C16-20, hydrocarbon-based chain;
(ii) fatty alcohol ethers of sugar(s) such as alkyl(poly)glycosides; and
(iii) mixtures thereof.

According to a particular embodiment, the nonionic surfactant(s) comprising one or more carbohydrate residues are chosen from fatty acid (poly)glyceryl esters of glucose or alkylglucose having a linear or branched, saturated or unsaturated, C6-C22, in particular C16-20, hydrocarbon-based chain, and mixtures thereof.

### Alkyl(Poly)Glycosides

The nonionic surfactants of alkyl(poly)glycoside type are notably represented by the general formula (III) below:

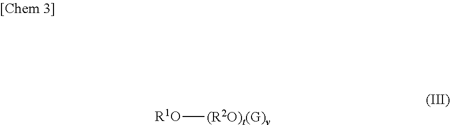

in which:
   R¹ represents a linear or branched alkyl or alkenyl radical including 6 to 24 carbon atoms and notably 8 to 20 carbon atoms, or an alkylphenyl radical of which the linear or branched alkyl radical includes 6 to 24 carbon atoms and notably 8 to 20 carbon atoms;
   R² represents an alkylene radical including 2 to 4 carbon atoms,
   G represents a sugar unit including 5 to 6 carbon atoms;
   t denotes a value ranging from 0 to 10 and in particular from 0 to 4;
   v denotes a value ranging from 1 to 15 and in particular from 1 to 4.

According to a particular embodiment, the alkyl(poly)glycoside surfactants are compounds of the formula described above in which:
R¹ denotes a linear or branched, saturated or unsaturated alkyl radical including from 8 to 20 carbon atoms,
R² represents an alkylene radical including 2 to 4 carbon atoms,
t denotes a value ranging from 0 to 3 and in particular equal to 0,
G denotes glucose, fructose or galactose, in particular glucose,
the degree of polymerization, i.e. the value of v, possibly ranging from 1 to 15 and in particular from 1 to 4; the mean degree of polymerization more particularly being between 1 and 2.

The glucoside bonds between the sugar units are generally of 1-6 or 1-4 type and in particular of 1-4 type.

As examples of alkyl(poly)glycosides, mention may be made of caprylyl/capryl glucoside, such as the product sold under the name Oramix CG 110L® by the company SEPPIC; decyl glucoside sold under the name Sorbithix L-100® by the company Applechem; arachidyl glucoside glucoside, optionally as a mixture with arachidyl alcohol and behenyl alcohol, sold, for example, under the name Montanov 202® by the company SEPPIC; cetylstearyl glucoside, optionally as a mixture with cetylstearyl alcohol, sold, for example, under the name Montanov 68MB® by the company SEPPIC, under the name Emulgade PL 68/50® by the company BASF and under the name Tego Care CG 90 MB® by the company Evonik Goldschmidt; cocoyl glucoside, such as the product sold under the name Lamesoft PO65® by the company BASF; octyldodecyl xyloside, sold, for example, under the name Fluidanov 20X by the company SEPPIC.

The alkyl(poly)glycoside may be used as a mixture with at least one fatty alcohol, notably a fatty alcohol containing from 6 to 24 carbon atoms and more particularly from 8 to 20 carbon atoms.

For example, it is possible to use in combination a fatty alcohol and an alkyl(poly)glycoside whose alkyl part is identical to that of the selected fatty alcohol.

The fatty alcohol/alkyl polyglycoside emulsifying mixtures as defined above are known per se. They are notably described in patent applications WO 92/06778, WO 95/13863 and WO 98/47610 and prepared according to the preparation processes indicated in these documents.

Among the fatty alcohol/alkyl(poly)glycoside mixtures, mention may be made of the products sold by the company SEPPIC under the name Montanov®, such as the following mixtures:
arachidyl alcohol and behenyl alcohol/arachidyl glucoside-Montanov 202®
cetylstearyl alcohol/cetylstearyl glucoside-Montanov 68MB®.

In a particular embodiment, the composition according to the invention comprises an alkyl(poly)glycoside surfactant chosen from caprylyl/capryl glucoside, arachidyl glucoside and mixtures thereof.

In a particular embodiment, the composition according to the invention does not comprise any decyl glucoside.

### Fatty Acid (Poly)Glyceryl Esters of Glucose or Alkylglucose

The fatty acid (poly)glyceryl esters of glucose or alkylglucose containing a linear or branched, saturated or unsaturated C6-C22 hydrocarbon-based chain are in particular fatty acid (poly)glyceryl esters of alkylglucose containing a linear, saturated C6-C22, in particular C16-20, more particularly C18, hydrocarbon-based chain.

Among the fatty acid (poly)glyceryl esters of glucose or alkylglucose containing a linear or branched, saturated or unsaturated, C6-C22 hydrocarbon-based chain, polyglyceryl-3-methylglucose distearate is most particularly preferred.

Among the commercial products, mention may be made of the product sold by the company Evonik Goldschmidt under the name Tego Care 450.

The nonionic surfactant(s) comprising one or more carbohydrate residues may be present in the composition according to the invention in a content ranging from 0.01% to 10% by weight, in particular in a content ranging from 0.05% to 8% by weight, more particularly in a content ranging from 0.1% to 5% by weight relative to the total weight of the composition, even more particularly in a content ranging from 0.2% to 3% by weight relative to the total weight of the composition.

A composition according to the invention may comprise one or more endolysins according to the invention and one or more nonionic surfactants comprising one or more carbohydrate residues according to the invention in a mass ratio of endolysin(s)/nonionic surfactant(s) comprising one or more carbohydrate residues of between 0.0001 to 0.5, in particular between 0.0002 and 0.1, more particularly between 0.0004 and 0.05, even better still between 0.0006 and 0.02.

### C6-C30 Fatty Acid Alkanolamides

The composition according to the invention may also contain at least one nonionic surfactant chosen from C6-C30 fatty acid alkanolamides.

Such surfactants may be chosen from mono- and di-alkanolamides of formula (IV):

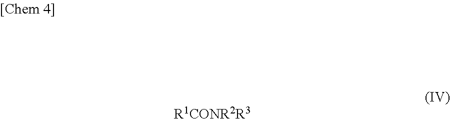

in which:
R¹ is a linear or branched, saturated or unsaturated hydrocarbon-based group containing from 6 to 30 carbon atoms,
R² and R³, independently, are hydrogen or a linear or branched, saturated or unsaturated alkanol group containing from 1 to 10 carbon atoms, on condition that only one from among R² and R³ is hydrogen.

As examples of surfactants of this type, mention may be made of lauric acid monoethanolamide, lauric acid diethanolamide, lauric acid monopropanolamide, lauric acid monoisopropanolamide, myristic acid monoethanolamide, myristic acid diethanolamide, palmitic acid monoethanolamide, stearic acid monoethanolamide (stearamide MEA), oleic acid monoethanolamide, oleic acid diethanolamide, oleic acid monoisopropanolamide, coconut oil fatty acid monoethanolamide (cocamide MEA), coconut oil fatty acid monopropanolamide, coconut oil fatty acid monoisopropanolamide (cocamide MIPA), erucic acid diethanolamide, palm plant oil fatty acid monoethanolamide, and mixtures thereof.

According to a particular embodiment in formula (IV), R¹ is a linear or branched, saturated or unsaturated hydrocarbon-based group containing from 8 to 18 carbon atoms,
R² and R³, independently, are hydrogen or a linear or branched, saturated or unsaturated alkanol group containing from 2 to 5 carbon atoms, on condition that only one from among R² and R³ is hydrogen.

According to a particular embodiment, in formula (IV), R² is hydrogen, and R³ is a saturated linear or branched alkanol group containing 2 to 5 carbon atoms.

According to a particular embodiment, the appropriate C6-C30 fatty acid alkanolamide of formula (IV) is chosen from coconut oil fatty acid monoethanolamide (INCI: cocamide MEA or cocamide monoethanolamine), coconut oil fatty acid monoisopropanolamide (INCI: cocamide MIPA or cocamide monoisopropanolamine), and mixtures thereof.

Such products are commercially available: for example, coconut oil fatty acid monoethanolamide (cocamide MEA) sold under the name Comperlan® 100 by the company Cognis (BASF), coconut oil fatty acid monoisopropanolamide (cocamide MIPA) sold under the trade name Empilan® CIS by the company Innospec Active Chemicals.

According to a particular embodiment, said C6-C30 fatty acid alkanolamide is cocamide monoisopropanolamine.

The C6-C30 fatty acid alkanolamide according to the present invention may be present in an amount ranging from 0.5% to 10% by weight, more particularly from 0.1% to 5% by weight, relative to the total weight of the composition.

According to a particular embodiment, a composition according to the invention comprises an amount of less than 2% by weight, relative to the total weight of the composition, of fatty acid(s) that are solid at room temperature (25° C.); more particularly, a composition according to the invention comprises an amount of less than 1% by weight, relative to the total weight of the composition, of fatty acid(s) that are solid at room temperature (25° C.), or even is free of (0% by weight relative to the total weight of the composition) fatty acid(s) that are solid at room temperature (25° C.), and in particular does not comprise any stearic acid.

According to a particular embodiment, a composition according to the invention comprises an amount of less than 0.5% by weight, relative to the total weight of the composition, of benzoic acid and/or salts thereof, notably alkali metal or alkaline-earth metal benzoates such as sodium benzoate, of sorbic acid and/or salts thereof, notably alkali metal or alkaline-earth metal sorbates such as potassium sorbate, more particularly less than 0.1% by weight, or even is free of (0% by weight relative to the total weight of the composition) benzoic acid and/or salts thereof such as sodium benzoate, and sorbic acid and/or salts thereof such as potassium sorbate.

According to a particular embodiment, a composition according to the invention comprises an amount of less than 0.1% in total amount by weight, relative to the total weight of the composition, of carrageenan, gellan gum, scleroglucan gum, gum arabic, pectin, xanthan gum, guar gum such as hydroxypropyl guar, hydrogenated soybean lecithin, sodium alginate, polyacrylamidomethylpropanesulfonic acid, carbomer, cellulose, sodium polyacrylate, konjac gum, agar, and Caesalpinia spinosa gum; more particularly, according to one embodiment of the invention, the composition is free of (0% by weight relative to the total weight of the composition) carrageenan, gellan gum, scleroglucan gum, gum arabic, pectin, xanthan gum, guar gum such as hydroxypropyl guar, hydrogenated soybean lecithin, sodium alginate, polyacrylamidomethylpropanesulfonic acid, carbomer, cellulose, sodium polyacrylate, konjac gum, agar and Caesalpinia spinosa gum.

According to a particular embodiment, a composition according to the invention comprises an amount of less than 0.5% in total amount by weight, relative to the total weight of the composition, of anionic surfactant; more particularly, the composition is free of anionic surfactant (0% by weight relative to the total weight of the composition).

According to a particular embodiment, a composition according to the invention comprises an amount of less than or equal to 0.5% in total amount by weight, relative to the total weight of the composition, of cationic surfactant; more particularly, the composition is free of cationic surfactant (0% by weight relative to the total weight of the composition).

According to a particular embodiment, a composition according to the invention comprises an amount of less than or equal to 0.5% in total amount by weight, relative to the total weight of the composition, of amphoteric surfactant; more particularly, the composition is free of amphoteric surfactant (0% by weight relative to the total weight of the composition).

According to a particular embodiment, a composition according to the invention comprises an amount of less than or equal to 0.5% in total amount by weight, relative to the total weight of the composition, of zwitterionic surfactant, and more particularly is free of zwitterionic surfactant (0% by weight relative to the total weight of the composition).

According to a particular embodiment, a composition according to the invention comprises an amount of less than 0.5% in total amount by weight, relative to the total weight of the composition, of anionic, cationic, amphoteric and zwitterionic surfactant, more particularly an amount of less than 0.1% in total amount by weight, relative to the total weight of the composition, of anionic, cationic, amphoteric and zwitterionic surfactants, even more particularly an amount of less than 0.01% in total amount by weight, relative to the total weight of the composition, of anionic, cationic, amphoteric and zwitterionic surfactants; better still, according to a particular embodiment, the composition is free of anionic, cationic, amphoteric and zwitterionic surfactants (0% by weight relative to the total weight of the composition).

According to a particular embodiment, a composition according to the invention comprises less than 0.5% in total amount by weight, relative to the total weight of the composition, of glyceryl stearate citrate, alkyl sulfate such as sodium lauryl sulfate, alkyl ether sulfate such as sodium laureth sulfate, disodium cocoamphodiacetate, fatty acid polyglyceryl esters such as polyglyceryl-4 isostearate, polyglyceryl-4 diisostearate polyhydroxystearate sebacate, sodium stearate, glyceryl stearate citrate, alkyl sulfate such as sodium lauryl sulfate, alkyl ether sulfate such as sodium laureth sulfate, disodium cocoamphodiacetate, fatty acid polyglyceryl esters such as polyglyceryl-4 isostearate and polyglyceryl-4 diisostearate polyhydroxystearate sebacate, and sodium stearate.

According to a particular embodiment, a composition according to the invention comprises an amount of less than 1% by weight, relative to the total weight of the composition, of kaolin, perlite, titanium dioxide, talc, cellulose, boron nitride, maltodextrin and mica, and more particularly comprises an amount of less than 0.5% by weight, relative to the total weight of the composition, of kaolin, perlite, titanium dioxide, talc, cellulose, boron nitride, maltodextrin and mica, or even is free of (0% by weight relative to the total weight of the composition) kaolin, perlite, titanium dioxide, talc, cellulose, boron nitride, maltodextrin and mica.

According to a particular embodiment, a composition according to the invention comprises an amount of less than 0.1% by weight, relative to the total weight of the composition, of fatty acid that is solid at room temperature (25° C.), and notably does not comprise any stearic acid; of benzoic acid, and/or salts thereof, notably alkali metal or alkaline-earth metal benzoates such as sodium benzoate, of sorbic acid and/or salts thereof, notably alkali metal or alkaline-earth metal sorbate, including potassium sorbate; of carrageenan, gellan gum, scleroglucan gum, gum arabic, pectin, xanthan gum, guar gum such as hydroxypropyl guar, hydrogenated soybean lecithin, sodium alginate, polyacrylamidomethylpropanesulfonic acid, carbomer, sodium polyacrylate, konjac gum, agar and Caesalpinia spinosa gum; glyceryl stearate citrate, alkyl sulfate such as sodium lauryl sulfate, alkyl ether sulfate such as sodium laureth sulfate, disodium cocoamphodiacetate, fatty acid polyglyceryl esters such as polyglyceryl-4 isostearate and polyglyceryl-4 diisostearate polyhydroxystearate sebacate, and sodium stearate; kaolin, perlite, titanium dioxide, talc, cellulose, boron nitride, maltodextrin and mica.

A composition according to the invention may be in any presentation form normally used in the cosmetics field, for instance in the form of an aqueous gel, lotion, foam, micellar water, cream, paste or serum.

A composition according to the invention is preferentially suitable for topical administration. Thus, a composition according to the invention may comprise all the constituents usually employed in the envisaged topical application and administration.

A composition according to the invention may advantageously be in the form of an emulsion, notably obtained by dispersion of an aqueous phase in a fatty phase (W/O) or of a fatty phase in an aqueous phase (O/W), of liquid or semi-liquid consistency of the milk type, or of soft consistency, or alternatively of multiple emulsion (W/O/W or O/W/O). These compositions are prepared according to the usual known methods.

More particularly, a composition according to the invention may be intended for topical application and may preferably be in the form of an emulsion, preferably an oil-in-water emulsion. Preferably, such an emulsion is not intended to be rinsed off after application.

A composition according to the invention is preferentially intended to be applied to a skin. Preferably, the skin is the skin of the face, scalp, neckline, neck, arms or forearms, or even more preferably the skin of the face (in particular of the forehead, nose, cheeks and chin), neckline and neck.

The composition may alternatively be in the form of a face and/or body care or makeup product, and may be packaged, for example, in the form of a cream in a jar or a fluid in a tube or a pump bottle or a dropper bottle.

The composition according to the invention may be manufactured via any known process generally used in the cosmetics field.

The ingredients are mixed before forming, in the order and under conditions readily determined by a person skilled in the art.

According to a particular mode of the invention, other agents intended to embellish the appearance and/or texture of the skin may also be added to the composition according to the invention.

### Uses and Processes

According to one of its aspects, the present invention relates to the cosmetic use, notably the topical use, of a composition according to the invention for preventing and/or treating a skin disorder linked to colonization by a bacterium of the Staphylococcus aureus species, and in particular for preventing and/or treating acne and/or eczema in an individual in need thereof. According to yet another one of its aspects, the present invention relates to a non-therapeutic cosmetic process for caring for keratin materials, in particular the skin, comprising the topical application to these keratin materials of a composition according to the invention.

A skin may in particular be a skin presenting acne or at risk of presenting acne and/or a skin presenting eczema or at risk of presenting eczema.

The cosmetic uses and processes considered according to the invention are non-therapeutic. The cosmetic uses and processes of the invention are preferentially performed by topically administering a composition according to the invention.

Topical administration consists of the external application to the skin of cosmetic compositions according to the usual techniques for the use of these compositions.

By way of illustration, the cosmetic use or process according to the invention may be performed by topical, for example daily, application of at least one composition according to the invention, which may be formulated, for example, as a cream, gel, serum, lotion, emulsion or makeup-removing milk.

The application may be repeated for example once or twice a day over a day or more and generally over an extended period of at least 3 days, at least 4 weeks, or even 4 to 15 weeks, with one or more periods of stoppage, if necessary.

According to one embodiment, the application is daily (once a day) and generally over an extended period of at least 3 days, at least 4 weeks or even 4 to 15 weeks, with one or more periods of stoppage, if necessary.

According to one embodiment, the cosmetic treatment process according to the invention may comprise a single application.

Throughout the description, including the claims, the terms “between . . . and . . . ”, and “ranging from . . . to . . . ” should be understood as meaning limits included, unless otherwise specified.

The examples that follow illustrate the present invention without limiting the scope thereof. In the examples, unless otherwise specified, the temperature is room temperature (25° C.) and is expressed in degrees Celsius, and the pressure is atmospheric pressure.

### Examples

The different compositions tested below are described in Table 1 below.

TABLE 1

| | | Formula | Formula |
|---|---|---|---|
| | | with | comprising |
| | | endolysin | the |
| | Endolysin- | of sequence | endolysin |
| | free | SEQ | of sequence |
| | formula | ID NO: | SEQ ID NO: |
| | with | 10 without | 10 and |
| | filler X | filler X | filler X |
| | (g per | (g per | (g per |
| | 100 g) | 100 g) | 100 g) |
| Base formula: | qs | qs | qs |
| Preserving agent | | | |
| Hydroxypropylmethylcellulose | | | |
| Propylene glycol | | | |
| Water | | | |
| Endolysin of sequence 10 | — | 0.002565 | 0.002565 |
| Mineral or organic filler X | 1 to 3 | — | 1 to 3 |

Two of these compositions are compositions according to the invention, comprising at least one organic filler as defined below. Comparative compositions, outside the invention, comprising other organic or mineral fillers outside the invention were also tested. Thus, in each of the compositions tested, X is equivalent, respectively, to:

TABLE 2

| Composition | Nature of X |
|---|---|
| 1 | Native corn starch sold under the name Beauty- |
| | by-Roquette ST005 by the company Roquette |
| | (according to the invention) |
| 2 | N-Lauroyl-L-lysine sold under the name Amihope |
| | LL ® by the company Ajinomoto (according to |
| | the invention) |
| 3 | Kaolin (outside the invention) |
| 4 | Perlite (outside the invention) |
| 5 | Cellulose microspheres (outside the invention) |
| 6 | Titanium dioxide (outside the invention) |
| 7 | Talc (outside the invention) |
| 8 | Cellulose (outside the invention) |
| 9 | Mica (outside the invention) |

### Tables 1 and 2

A preserving agent was added to the systems so as to avoid contamination during storage and thus to perform the various stability tests.

The formulations were prepared in a beaker with magnetic stirring at room temperature. All the ingredients were poured into the beaker and kept stirring for 30 minutes. The various ingredients of these compositions are present in the usual contents for each of them in a cosmetic composition, in particular in a usual content for each of them allowing it to play its role in a composition according to the invention.

### Protocol (Experimental Conditions)

Preparation of the Working Culture of Staphylococcus aureus

Preparation of the working suspension of Staphylococcus aureus (S. aureus) from a lyophilizate ATCC 6538 (according to the recommendations of the standard NF EN 12353). The lyophilizate is rehydrated in Trypticase soy broth, plated on Trypticase soy agar (TSA plates) and incubated for 24 hours at 32.5° C. The cells are then recovered and resuspended in a commercial cryoprotectant solution with cryobeads for storage at −80° C. for a maximum of 14 months (long-term storage stock).

From a cryobead of this −80° C. stock, subculturing is performed on TSA slant agar which is then incubated for 24 hours at 32.5° C. to obtain the stock culture. This stock culture is stored at 4° C. for a maximum of 9 weeks. The working culture is obtained by subculturing the stock culture onto Trypticase soy agar and incubating it for 24 hours at 35° C. The working suspension is prepared by suspending the cells of this working culture in a tryptone salt diluent. This suspension is calibrated between 1 and 3×10⁸ CFU (colony forming units)/mL by measuring the absorbance at 620 nm.

Evaluation of the Antimicrobial Activity of the Samples Against S. aureus

The 10 formulations of Table 1 above were tested.

At T=1 week of the manufacture of the formulations, 20-gram aliquots of each formulation are inoculated with 0.2 ml of a calibrated suspension of S. aureus. After homogenization, the microorganism content in the product represents a concentration of S. aureus of 10⁶ CFU per gram of product, i.e. a 1% inoculation of a suspension of 10⁸ CFU per mL (the inoculum content is determined by spreading the suspension on Trypticase soy agar plates and incubating for 24 hours at 35° C.). After t=30 minutes and t=60 minutes of contact at room temperature (20° C.±3° C.), 1 gram of the mixture is weighed and 9.0 mL of Eugon LT100 supp broth are then added, followed by mixing until fully homogenized. This mixture is then serially diluted in Eugon LT100 supp broth to 1/100 dilution.

The dilutions are spread on Trypticase soy agar plates and incubated at 35° C. for 48 hours until the surviving colonies of S. aureus are counted.

The antimicrobial activity on S. aureus is expressed in logarithmic abatement relative to the initial content and associated with the activity of the active compound, which is the endolysin of sequence SEQ ID NO: 10 by comparing the counts of surviving S. aureus in formulations with and without the endolysin of sequence SEQ ID NO: 10.

This method is an adaptation of the challenge test method described in the standard “ISO 11930 Cosmetics—Microbiology—Evaluation of the antimicrobial protection of a cosmetic product”.

### RESULTS AND CONCLUSIONS

The results are expressed as logarithmic Staphylococcus aureus abatement relative to the inoculated content. The abatements are thus between 0 log (no antimicrobial activity observed under the test condition i.e. complete loss of activity of the endolysin of sequence SEQ ID NO: 10) and −5.4 log (maximum reduction observable under the test conditions, i.e. maintenance of endolysin activity under the test condition). The formula tested is thus particularly active when the values are close to −5.4.

Evaluation of the antimicrobial activity of the endolysin of sequence SEQ ID NO: 10 is taken at T=1 week from the date of manufacture of the formulations. This evaluation is performed according to the protocol described above, i.e. measurement of the activity of the endolysin in the formulation after t=30 minutes of contact and 1 hour of contact with S. aureus/formulation.

The results are given in Table 3 below.

TABLE 3

| | | Content of | | | |
|---|---|---|---|---|---|
| | | endolysin | | | |
| | Content | of sequence | | Log | Log |
| | of filler | SEQ ID | | Reduction | Reduction |
| Formulations | X (%) | NO: 10 (%) | pH | t30 min | t1 h |
| Formulation | 0 | 0 | 7.0 | 0.1 | 0.0 |
| with endolysin | | 0.002565 | 7.0 | −5.4 | −5.4 |
| and without | | | | | |
| filler X | | | | | |
| 1 Native corn | 1.5 | 0 | 5.7 | 0.1 | 0 |
| starch | | 0.002565 | 6.4 | −4.3 | −5.3 |
| 2 Lauroyl | 3 | 0 | 5.4 | 0.1 | 0 |
| lysine | | 0.002565 | 6.8 | −4.5 | −5.3 |
| 3 Kaolin | 3 | 0 | 5.5 | 0 | 0 |
| | | 0.002565 | 5.5 | 0.1 | 0.1 |
| 4 Perlite | 3 | 0 | 6.6 | 0 | 0.1 |
| | | 0.002565 | 6.8 | 0.1 | 0.1 |
| 5 Cellulose | 3 | 0 | 5.8 | 0.1 | 0 |
| microspheres | | 0.002565 | 6.2 | 0.2 | 0 |
| 6 Titanium | 1 | 0 | 6.5 | 0.1 | 0.1 |
| dioxide | | 0.002565 | 6.8 | 0.1 | 0.1 |
| 7 Talc | 3 | 0 | 9.5 | 0.1 | 0.1 |
| | | 0.002565 | 8 | 0.1 | 0.1 |
| 8 Cellulose | 3 | 0 | 5.7 | 0.1 | 0.1 |
| | | 0.002565 | 5.2 | 0 | 0 |
| 9 Mica | 3 | 0 | 8.3 | 0.1 | 0.1 |
| | | 0.002565 | 7.4 | 0.1 | 0.1 |

### Table 3

The results show that the combination of an organic filler such as native corn starch or N-lauroyl-L-lysine according to the invention with the endolysin of sequence SEQ ID NO: 10 allows the antibacterial activity against S. aureus of endolysin to be maintained—as shown by the Log reduction results in Table 3—compared with the performance obtained with other organic or mineral fillers.

The results show that the endolysin activity is greater than 99.99% (Log reduction <−4) in killing S. aureus colonies at 1 week/25° C. for the system comprising corn starch or N-lauroyl-L-lysine (30 min and 1 h contact). Native corn starch and N-lauroyl-L-lysine are thus capable of maintaining the antibacterial performance against S. aureus of endolysin over time, as shown by the Log reduction results in Table 3.

Comparative preparations with the other organic or mineral fillers tested show significantly lower activity at 1 week under similar conditions.

Surprisingly, the results thus show that the combination of endolysin with an organic filler such as native corn starch or N-lauroyl-L-lysine allows better maintenance of antibacterial activity against S. aureus over time, relative to the combination of endolysin with other organic or mineral fillers.

1. A composition comprising, in a physiologically acceptable medium:
i. at least one endolysin; and
ii. at least one organic filler chosen from an unmodified starch, an N-acylamino acid, salts thereof and mixtures thereof.

2. The composition according to claim 1, in which the endolysin is an endolysin derived from a Staphylococcus aureus phage.

3. The composition according to claim 1, in which the endolysin comprises a first protein sequence comprising a domain for binding to the cell wall of species of the genus Staphylococcus.

4. The composition according to claim 3, in which the first protein sequence is derived from the endolysin of the Φ2638a bacteriophage of S. aureus.

5. The composition according to claim 3, in which the first protein sequence comprises a protein sequence comprising at least 80% sequence identity with the amino acid sequence of reference SEQ ID NO: 1.

6. The composition according to claim 1, in which the endolysin comprises a protein sequence comprising at least 80% sequence identity with an amino acid sequence chosen from the group consisting of the amino acid sequences of references SEQ ID NO: 3 and SEQ ID NO: 4.

7. The composition according to claim 3, in which said endolysin also comprises a heterologous protein sequence.

8. The composition according to claim 6, in which the heterologous protein sequence comprises a lytic domain, said lytic domain comprising a second and a third protein sequence, said second protein sequence comprising an endopeptidase M23 domain and said third protein sequence comprising an amidase domain.

9. The composition according to claim 8, in which said second and third protein sequences are derived, independently of each other, from an enzyme chosen from the group consisting of the endolysin from Φ2638a bacteriophage of S. aureus and the lysostaphin from S. simulans.

10. The composition according to claim 8, in which said second protein sequence comprises at least 80% sequence identity with the amino acid sequence of reference SEQ ID NO: 6 and said third protein sequence comprises at least 80% sequence identity with the amino acid sequence of reference SEQ ID NO: 8.

11. The composition according to claim 1, in which the endolysin comprises a protein sequence comprising at least 80% sequence identity with an amino acid sequence chosen from the group consisting of the amino acid sequences of references SEQ ID NO: 10 and SEQ ID NO: 11.

12. The composition according to claim 1, in which the endolysin is present in a content ranging from 0.0001% to 0.1% by weight relative to the total weight of the composition.

13. The composition according to claim 1, in which the unmodified starch is chosen from corn starches, rice starches, potato starches and mixtures thereof.

14. The composition according to claim 1, in which the unmodified starch is a corn starch.

15. The composition according to claim 1, in which the unmodified starch is present in the composition in a content ranging from 0.1% to 10% by weight relative to the total weight of the composition.

16. The composition according to claim 1, in which the N-acylamino acid is lauroyl lysine.

17. The composition according to claim 1, in which the N-acylamino acid is present in the composition in a content ranging from 0.1% to 15% by weight relative to the total weight of the composition.

18. The composition according to claim 1, the composition being suitable for topical administration.

19. The composition according to claim 1, for use in the prevention and/or treatment of a skin disorder linked to colonization by a bacterium of the species Staphylococcus aureus in an individual in need thereof.

20. The composition according to claim 19, in which the composition is suitable for topical administration.

21. A non-therapeutic cosmetic process for caring for keratin materials comprising the topical application to said keratin materials of a composition according to claim 1.


SEQUENCE LISTING (13 sequences)

SEQ ID NO: 1 - protein, 93 aa, synthetic construct
WKQNKDGIWYKAEHASFTVTAPEGIITRYKGPWTGHPQAGVLQKGQTIKYDEQKFDGHVWVSWETFEGETVYMPVRTWDAKTGKVGKLWGEIK

SEQ ID NO: 2 - DNA, 285 bp, synthetic construct
  source 1..285: mol_type = other DNA
tggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 3 - protein, 486 aa, synthetic construct
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 4 - protein, 498 aa, synthetic construct
MRGSHHHHHHGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 5 - DNA, 1461 bp, synthetic construct
  source 1..1461: mol_type = other DNA
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 6 - protein, 140 aa, synthetic construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGY

SEQ ID NO: 7 - DNA, 420 bp, synthetic construct
  source 1..420: mol_type = other DNA
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatat

SEQ ID NO: 8 - protein, 169 aa, synthetic construct
NKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDG

SEQ ID NO: 9 - DNA, 510 bp, synthetic construct
  source 1..510: mol_type = other DNA
ggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggt

SEQ ID NO: 10 - protein, 505 aa, synthetic construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 11 - protein, 517 aa, synthetic construct
MRGSHHHHHHGSAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 12 - DNA, 1518 bp, synthetic construct
  source 1..1518: mol_type = other DNA
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcttacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 13 - protein, 12 aa, synthetic construct
MRGSHHHHHHGS